(19) United States
(12) Patent Application Publication
(10) Pub. No.: US 2022/0357312 A1
(43) Pub. Date: Nov. 10, 2022

(54) METHODS FOR DETERMINING ANTIBODY AFFINITY AND BINDING KINETICS USING VLPS OR LIVE VIRUSES ATTACHED TO BIOSENSORS

(71) Applicant: Takeda Vaccines, Inc., Cambridge, MA (US)

(72) Inventors: Isamu TSUJI, Cambridge, MA (US); Hansi DEAN, Cambridge, MA (US); Michael EGAN, Nanuet, NY (US)

(73) Assignee: Takeda Vaccines, Inc., Cambridge, MA (US)

(21) Appl. No.: 17/766,038

(22) PCT Filed: Oct. 2, 2020
(86) PCT No.: PCT/US2020/053964

§ 371 Date: Apr. 1, 2022

(30) Foreign Application Priority Data
Oct. 2, 2019 (EP) 19201211.0

(51) Int. Cl. G01N 33/50; C07K 16/10; C12N 7/00; C07K 14/005

(52) U.S. Cl. CPC G01N 33/502; C07K 16/1081; C12N 7/00; C07K 14/005; C07K 2317/76; C07K 2317/92; C12N 2770/16023; C12N 2770/24123

(57) ABSTRACT

The present invention relates to methods for determining affinity, binding kinetics and/or concentration of an antibody or of an antibody mixture specific for a virus using virus-like particles (VLPs) and/or live viruses or inactivated viruses attached to biosensors. Further, the present invention relates to the VLPs and live or inactivated viruses attached to biosensors and methods for producing them.

### TECHNICAL FIELD

The present invention relates to methods for determining affinity, binding kinetics and/or concentration of an antibody or of an antibody mixture specific for a virus using virus-like particles (VLPs) and/or live viruses or inactivated viruses attached to biosensors. Further, the present invention relates to the VLPs and live or inactivated viruses attached to biosensors and methods for producing them.

### BACKGROUND OF THE INVENTION

Antibody affinity maturation is the process whereby the immune system generates antibodies of higher affinities during a response to antigen through somatic hypermutation. Antibody somatic hypermutation takes place in germinal centers after exposure to antigen, either by infection or immunization. B cells in the germinal centers express enzymes which insert point mutations throughout the Ig heavy and light chains. The repertoire of mutated B cells is then selected and enriched for high affinity of the antibody to its cognate antigen. Iterative rounds of selection and proliferation of somatically mutated clonal variants result in a population of antibodies that are enriched for higher affinity binders, based on successive accumulation of somatic mutations over time. Collectively, antibodies with increased affinities after antigen exposure contribute to an overall increase in polyclonal antibody avidity.

The importance of antibody affinity maturation to effective antiviral responses is well established. For example, HIV antibody affinity correlates with neutralization potency and breadth. Affinity maturation of B cells specific for conserved epitopes after sequential exposure to infection is required for protection from re-infection by diverse influenza viruses and is required to generate mAbs of sufficient potency for Ebola virus therapy. A common theme of successful antiviral immunity is induction of high affinity functional antibodies to conserved epitopes, in the context of abundant ineffective immune responses to variable viral epitopes. Information on antibody affinity maturation during dengue infections is limited, but available studies point to the potential role of affinity matured antibodies in resistance to post-secondary dengue infections. After a second dengue infection, neutralizing antibodies were boosted to the previous infecting serotype and cross-neutralizing antibodies to all serotypes increase. Over time, neutralizing antibody response magnitude decreases, but breadth of neutralizing antibodies is maintained, therefore the quantity of antibody decreases, but the quality of neutralizing antibodies is stably altered. After an individual has had 2 dengue infections, a third infection is rarely symptomatic, suggesting that immune responses elicited by a second dengue infection are protective. Repeated DENV infections have been shown to increase monoclonal and polyclonal antibody avidity and increased neutralization potency.

Affinity maturation leads to antibodies of higher affinity and avidity, required for optimal antiviral functions, including virus neutralization and antibody-dependent cell-mediated cytotoxicity. Sometimes, an increase of antiviral immunity in an individual is mainly due to the activity of a single affinity matured antibody, but it is generally believed to be mediated by the combined effect of multiple affinity matured antibodies that are present in the circulation.

The titer of neutralizing antibodies in serum is the most common measure of antibody responses to vaccination and infection. However, neutralizing antibodies do not always correlate with vaccine efficacy and neutralization assays do not measure all antibody effector functions. The degree of affinity maturation driven by vaccination or natural infection is an important parameter to be measured.

It is in particular important to measure antibodies directed to particulate antigens, since said particulate antigens preserve the conformational and quaternary epitopes being the targets of many potent neutralizing antibodies. For example, it is known that for Dengue virus, neutralizing antibodies generally bind only to the complete virus or virus-like particles (VLPs), but not to the isolated envelope proteins or peptides. Metz et al. reported weak reactivity of quaternary epitope antibodies to recombinant envelope proteins, because the Envelope-protein formed monomers, PLOS Neglected tropical diseases 12 (2018) e0006793.

In a study by Lau et al., J. Clin. Virol. 69 (2015), 63-67 the antibody avidity of antibodies to dengue virus type 2 virions was determined. The antibodies were detected by an enzyme-linked immunosorbent assay (ELISA) conducted in the presence of chaotropic reagents such as 8M urea in order to reduce the non-specific binding The described ELISA assay could not be reproduced by other laboratories.

While the described ELISA method provides high throughput, the method lacks accuracy. The method is prone to select strong binding antibodies over antibodies with weaker binding affinity.

Thus, there remains a need for an assay suitable for the determination of the binding affinity or avidity of antibodies specific for viruses. In particular, the assay shall be suitable for the determination of the binding affinity or avidity of antibodies or antibody mixtures directed to particulate antigens preserving conformational and quaternary epitopes.

### SUMMARY OF THE INVENTION

The technical problems underlying the invention are solved by the provision of the subject-matter as defined in the claims.

According to a first aspect is provided a method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

According to a second aspect is provided a method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

According to a third aspect is provided a method for determining the avidity and/or affinity over time of an antibody or antibody mixture produced after immunization of a human subject with a virus vaccine comprising the following steps:
a) obtaining serum samples from said subject at different time points after immunization;
b) purifying the antibody or antibody mixture from the serum samples by affinity chromatography using Protein A, Protein G, Protein A/G, Protein L or anti-human IgG;
c) determining the avidity and/or affinity of the antibody or the antibody mixture specific for the virus in accordance with the method according to the present invention; and
d) assessing the avidity and/or affinity of the antibody or antibody mixture as a function of time.

According to a fourth aspect is provided a method for preparing a virus-like particle (VLP) attached to a biosensor, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

According to a fifth aspect is provided a VLP attached to the biosensor which is obtainable by the method of the present invention.

According to a sixth aspect is provided a method of preparing a live virus or an inactivated virus attached to a biosensor, wherein the method comprises attaching said live virus or said inactivated virus to the biosensor by hydrophobic interaction of said live virus or said inactivated virus with a capture reagent linked to the surface of the biosensor.

According to a seventh aspect is provided the live virus or inactivated virus attached to the biosensor.

Surprisingly, the inventors have found that methods for detecting and monitoring biological interactions in real-time such as surface plasmon resonance (SPR) technology or biolayer interferometry (BLI) can be successfully applied in the determination of binding parameters such as the avidity index of antibodies directed to particulate antigens including quaternary and conformational epitopes. According to the invention the particulate antigen, in particular the live virus or the VLP, is attached to the biosensor. This allows the analysis of complex antibody mixtures from patient samples or vaccinated individuals.

Kumar et al., Biosensors 6 (2016), pages 1 to 16 discloses the application of SPR technology for the analysis of binding of live viruses to a biosensor being modified with a glycan, a virus-specific antibody or an aptamer. Such a system, however, does not allow the assessment of the binding parameters of antibodies from samples or vaccinated individuals. The prior art therefore rather teaches away from the direct coupling of the live virus or the VLP to the biosensor surface.

The present inventors have further found that the avidity index, i.e. the ratio of response/dissociation rate (k_{off}) for antibodies or antibody mixtures from vaccinated individuals, calculated from the SPR or BLI measurement data is useful for assessing the efficacy of the vaccine in vivo. Thus, the SPR or BLI measurement can be used for an in vitro assessment of the affinity maturation of the antibodies in the vaccinated individuals over time.

### BRIEF DESCRIPTION OF THE DRAWINGS

FIG. 1 shows the plate layout.

FIG. 2 shows the biosensor plate layout. SA means streptavidin biosensor.

FIG. 3 shows an SDS Page analysis of anti-DENV Ab purified from DEN203 sera sample.

FIG. 4 shows the optimization of Dengue VLP biotinylation.

FIG. 5 shows the Biosensor image of dengue vaccine immunized patient, ID10122005.

FIG. 6 shows the Biosensor image of dengue vaccine immunized patient, ID1044010.

FIG. 7 shows the changes in avidity of DENV1 specific antibodies for immunized patients.

FIG. 8 shows the changes in avidity of DENV2 specific antibodies for immunized patients.

FIG. 9 shows the changes in avidity of DENV3 specific antibodies for immunized patients.

FIG. 10 shows the changes in avidity of DENV4 specific antibodies for immunized patients.

FIG. 11 shows the results of avidity assay of Dengue live virus serotype 3. FIG. 1A relates to IgG from negative control sera 250 ug/mL. FIG. 1B relates to IgG from 1081012 250 ug/mL. FIG. 1C relates to IgG from positive control sera 250 ug/mL. FIG. 1D relates to IgG from 1082004 250 ug/mL. FIG. 1E relates to IgG from 1073001 D90 250 ug/mL.

FIG. 12 shows the results of avidity assay of Zika virus specific antibodies containing sera; ID 55 51766 P2 (Negative sera) ID 1043-TDS-0485 (Positive sera).

FIG. 13 shows the results of avidity assay of Noro VLP GII.2 Sydney 2012 sera. BRH1540413 (Negative sera) BRH1439733 (Positive sera)

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used, it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

According to a first aspect is provided a method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

In one embodiment, said method does not comprise a signal enhancing step. In particular, said method does not comprise the use of a secondary antibody coupled to a label or enzyme. Thus, in said embodiment the method is advantageously simple and efficient.

According to a second aspect is provided a method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by surface plasmon resonance or biolayer interferometry; and
d) calculating the affinity, binding kinetics and/or concentration of the antibody or the antibody mixture specific for the virus from the measurement data in steps b) and c).

“Virus” herein means any virus including double-stranded and single-stranded DNA viruses, and double and single-stranded RNA viruses. Preferably, the virus may be a flavivirus or a calicivirus. Among the flaviviruses, preferred are Dengue virus, Japanese encephalitis virus, Tick-borne encephalitis virus, West Nile virus, Yellow fever and Zika virus. Particularly preferred, the flavirus may be a Dengue virus or a Zika virus. Most preferred, the virus may be a dengue virus subtype selected from DENV-1, DENV-2, DENV-3 and DENV-4. Among the caliciviruses, Norovirus is preferred. In one embodiment, the live virus or inactivated virus is not influenza virus. In another embodiment, the live virus or inactivated virus is not an Enterovirus 71 F-particle.

The virus may be a live virus capable of replication. The virus may be a wild-type or a live attenuated virus. “Wild-type virus” refers to the phenotype of the typical form of a virus as it occurs in nature. “Live attenuated virus” refers to a weakened, less vigorous virus as compared to the wild-type form of the virus which is still viable and able to replicate. An attenuated virus may be used to produce a vaccine that is capable of stimulating an immune response.

Attenuation may be achieved by serial passaging of the virus in a foreign host such as in tissue culture, embryonated eggs or live animals. Alternatively, attenuation may be performed by chemical agents.

The viruses include recombinant variants such as chimeric viruses. The term “recombinant virus” is generally used for a genetically modified virus that carries nucleotide sequences from a viral or non-viral species which are not present in the wild-type virus. A “chimeric virus” is generally used for a recombinant virus that consists of a combination of the genomes of two parent viruses and which may display biological properties characteristic for both parent viruses.

The virus may also be an inactivated virus. Virus inactivation renders the viruses inactive, or unable to infect. Suitable methods for virus inactivation include solvent/detergent inactivation, treatment with chemical agents such as formalin and beta-propiolactone, heating and/or acidic pH inactivation. Inactivation methods are known to the person skilled in the art.

“Antibody or antibody mixture specific for a virus” herein includes antibodies of any source or synthetically prepared antibodies. The antibody may be a human or animal antibody. Preferably, the antibody is a human antibody. The antibody may be of any subtype including IgG and IgM, with IgG being preferred. The antibody may be generated in vitro or in vivo. The antibodies may be generated by immunization of individuals using vaccines comprising a live attenuated virus, an inactivated virus and/or a virus like particle (VLP) or viral proteins or peptides thereof. The vaccine may further include adjuvants known in the art.

The antibodies or antibody mixture may also be obtained from samples from virus infected patients. Samples from whole blood or serum are preferred, most preferred the samples are from serum. The obtained sample may be purified before the use in the method according to the invention. Suitable antibody purification methods such as ion exchange chromatography, affinity chromatography or hydrophobic chromatography are known to the person skilled in the art.

“Affinity” describes the strength of the interaction between two biomolecules such as an antigen and an antibody specific for the antigen. Extremely strong interactions can be in the picomolar range, while weak interactions can be in the millimolar range.

The dissociation constant (K_{D}) is the concentration of analyte at which half of all binding sites are occupied (at equilibrium conditions).

“Binding kinetics” relates to the rate at which the binding sites at a molecule such as an antibody are occupied with the ligand molecules such as antigens, i.e. the formation of the binding complex (association rate kₒₙ) and to the rate at which the ligand molecules are released from the binding sites, i.e. the dissociation of the binding complex (dissociation rate k_{off}). According to a preferred embodiment the association rate kₒₙ is measured when the binding sites attached to the biosensor are contacted with a solution containing the ligand molecules. According to a preferred embodiment the dissociation rate k_{off} is measured when the biosensor with the binding complex is removed from the above solution and introduced into a solution which does not contain the ligand molecules such as a buffer solution.

“Concentration” is the abundance of a constituent such as an antibody in a mixture divided by the total volume of the mixture. Preferably, the concentration is the molar concentration defined as the amount of a constituent nᵢ (in moles) divided by the volume of the mixture.

A “virus-like particle (VLP)” closely resembles a virus, but is non-infectious, since it does not contain genetic material. The VLP can be naturally occurring or synthesized through the individual expression of viral structural proteins, which can then self-assemble into the virus-like structure. Combinations of structural capsid proteins can be used to create recombinant VLPs. VLPs have been produced from components of a wide variety of virus families. VLPs can be produced in multiple cell culture systems including bacteria, mammalian cell lines, insect cell lines, yeast and plant cells. For a review on VLPs reference is made to Zeltins, Mol. Biotechnology 53 (2013), 92-107. VLPs can also be commercially obtained from companies such as the company Native Antigen. In one embodiment, the VLP is not derived from Norovirus.

“Biosensors” are devices used to detect the presence or concentration of a biological analyte, such as a biomolecule, a biological structure or a microorganism. Biosensors consist of three parts: a component that recognizes the analyte and produces a signal, a signal transducer, and a reader device. As used herein biosensors are suitable for use in connection with surface plasmon resonance (SPR) or biolayer interferometry (BLI) devices.

SPR is the resonant oscillation of conduction electrons at the interface between negative and positive permittivity material stimulated by incident light. Unlike many other immunoassays, such as ELISA, an SPR immunoassay is label free in that a label is not required for detection of the analyte. Additionally, the measurements on SPR can be followed in real-time allowing the monitoring of individual steps in sequential binding events. Useful systems in accordance with the present invention include Biacore® and IBIS® SPR systems.

BLI is a label-free technology for measuring biomolecular interactions. It is an optical analytical technique that analyzes the interference pattern of white light reflected from two surfaces: a layer of immobilized protein on the biosensor tip, and an internal reference layer. The binding between a ligand immobilized on the biosensor tip surface and an analyte in solution produces an increase in optical thickness at the biosensor tip, which results in a wavelength shift, Δλ, which is a direct measure of the change in thickness of the biological layer. Interactions are measured in real time, providing the ability to monitor binding specificity, rates of association and dissociation, or concentration, with high precision and accuracy; see Abdiche et al., Anal. Biochemistry 377 (2008), 209-217. Useful BLI systems for use in the present invention are the Pall-Fortebio® Octet® systems and the Pall-Fortebio® Blitz® systems. The Octet© system is preferred.

The biosensors suitable in connection with SPR or BLI can be in array format. Useful Biosensors with affinity surfaces for proteins or peptides are commercially available e.g. from the company ForteBio. Currently, biosensors with the following surface modifications are available: aminopropylsilane, amine reactive 2G, super streptavidin, anti-human Fc-capture, anti-mouse Fc-capture, streptavidin, anti-human IgG Fc, anti-murine IgG-Fv, anti-Penta-His, anti-His, Protein A, Protein G, Protein L, anti-human Fab-Ch1 2^{nd} generation, anti-GST and Ni-NTA (company Fortebio).

“Providing a VLP attached to biosensor” or “providing a live virus or inactivated virus attached to a biosensor” herein means that the VLP or the live virus or inactivated virus is immobilized on the surface of the biosensor by hydrophobic interactions or by covalent linkage. The immobilization can be direct or indirect such as mediated by binding partners.

For attaching a VLP to the biosensor any of the following is preferred:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

The pair of binding molecules is preferably selected from biotin/streptavidin; ligand/receptor; antigen/antibody; antibody/Protein A or Protein G; sugar/lectin; His-tag/Ni and sense/antisense oligonucleotides, particularly preferred the pair of binding molecules is biotin/streptavidin. Preferably, one member of said binding pairs is linked to the VLP by an activated moiety. Techniques for coupling of binding molecules to peptides and proteins are well-known in the art. Peptide coupling reagents include phosphonium reagents, uranium reagents, carbodiimide reagents, imidazolium reagents, organophosphorous reagents, acid halogenating reagents, chloroformate, pyridinium and other coupling reagents (see review article Han and Kim, Tetrahedron 60 (2004), 2447-2467).

Most preferred the VLP is biotinylated and the biosensor has streptavidin attached to its surface. Alternatively, it is preferred that the VLP is covalently linked to a biosensor having an amine-reactive surface such as the amine-reactive 2G biosensor commercially available from the company ForteBio.

For attaching the live virus or inactivated virus to the biosensor it is preferred that the attachment is mediated by hydrophobic interaction of the live virus or inactivated virus with a capture reagent linked to the surface of the biosensor. The capture reagent for attaching the live virus or the inactivated virus to the biosensor is preferably aminopropylsilane. In one embodiment, the live virus or the inactivated virus is not attached to to the biosensor via a biotinylated sialic acid polymer.

“Contacting the VLP attached to the biosensor with a first solution containing the antibody or antibody mixture specific for the virus such that the antibody or antibody mixture binds to the VLP attached to the biosensor” herein means that the antibody solution is contacted with the first solution under pH and salt conditions which allow the binding of the antibody to the VLP attached to the biosensor. Generally, the antibody will be present in a buffer system known in the art. Suitable buffers may be phosphate-buffered saline (PBS) or Tris-buffered saline (TBS). As outlined above for the VLP attached to the biosensor, the live virus attached to the biosensor or the inactivated virus attached to the biosensor may bind to the antibody or the antibody mixture specific for the virus under suitable pH and salt conditions. Generally, a buffer may also be used herein.

“Measuring the association of the binding complex” and “measuring the dissociation of the binding complex” herein includes the use of surface plasmon resonance (SPR) technology or biolayer interferometry (BLI) technology to measure the association and/or dissociation of the binding complex. For the present invention, the measuring by BLI is preferred. As discussed above, in BLI the association of the binding complex produces an increase in optical thickness at the biosensor tip which results in a measurable wavelength shift. In BLI the dissociation of the binding complex produces a decrease in optical thickness at the biosensor tip which results in a measurable wavelength shift. Thus, by recording the wavelength shift the association of the binding complex and/or the dissociation of the binding complex can be measured. The measurement is performed in real time, i.e. the association and/or dissociation can be followed over time.

“Calculating the affinity, binding kinetics and/or concentration of the antibody or the antibody mixture specific for the virus from the measurement data” herein means that the data obtained from the measurements using SPR or BLI are processed. Affinity calculations comprise the determination of the dissociation constant (K_{d}) or of the equilibrium constant (K_{eq}) for the binding of a ligand such as an antibody to a receptor such as a virus or VLP. Affinity calculations further include calculations known to the person skilled in the art for determining the effect of inhibitor binding. The influence of single and multiple binding sites may be calculated using e.g. the Scatchard Plot and the Hill Plot.

Binding kinetics calculations include the determination of the association rate (kₒₙ) and the dissociation rat (k_{off}) as outlined below. Further, binding kinetics calculations comprise calculations of the binding process such as single-step and two-step bimolecular binding processes.

These calculations can be performed by using commercially available software. Suitable software includes Octet Data Analysis Software from the company Fortebio.

In a further aspect a method is provided for determining the avidity and/or affinity over time of an antibody or antibody mixture produced after immunization of a human subject with a virus vaccine comprising the following steps:
a) obtaining serum samples from said subject at different time points after immunization;
b) purifying the antibody or antibody mixture from the serum samples by affinity chromatography using Protein A, Protein G, Protein A/G, Protein L, or anti-human IgG;
c) determining the avidity and/or affinity of the antibodies for the virus as a function over time in accordance with the method described herein.

In a preferred embodiment, the method comprises the following steps:
a) purifying the antibody or antibody mixture from the serum samples obtained from said subject at different time points after immunization by affinity chromatography using Protein A Protein G, Protein A/G or Protein L; and
b) determining the avidity and/or affinity of the antibodies for the virus as a function over time in accordance with any of the embodiments of the method according to the invention.

“Purifying antibodies or antibody mixtures from the serum samples by affinity chromatography using Protein A, Protein G, Protein A/G, Protein L or anti-human IgG” generally comprises the binding of the sample containing the antibodies or antibody mixture to the Protein A, Protein G, Protein A/G, Protein L or anti-human IgG matrix, the washing of the matrix with the bound antibody or antibody mixture and the elution of the bound antibody or antibody mixture from the matrix. Suitable conditions for binding of the antibody sample to the matrix include using a buffer at a pH from 7 to 8, wherein the buffer is preferably physiologically buffered. The washing can be done using phosphate-buffered saline. For the elution an acidic elution buffer, e.g. 0.1M glycine-HCL, pH 2.8 may be used. After elution from the matrix, the purified antibody sample is neutralized. Neutralization of the eluted samples can be done e.g. using a 1M Tris-HCL (pH 8.0) buffer.

Preferably, the method determines the avidity index of the antibody or antibody mixture from serum samples obtained after different points of time after immunization.

During the maturation of the humoral immune response, there is an antibody selection process that results in synthesis of antibodies with increased antigen-antibody association strength.

“Avidity” refers to the accumulated strength of multiple affinities of individual non-covalent binding interactions, such as between an antibody and its antigen. Calculations for avidity may include Scatchard plots. Another measure for avidity may be the avidity index.

“Avidity index” as used herein is calculated as follows: Avidity index=antibody response/k_{off}, wherein k_{off} is the dissociation rate of the complex. The unit of k_{off} is s⁻¹. Antibody response herein means the amount of specific antibody generated in reaction to immunization with a given antigen. The amount of antibody may be determined by BLI or SPR. Alternatively, the amount of antibody may be measured using ELISA assays.

Generally, antibodies produced at an early stage during primary response to an infection have lower antigen avidity than those produced at a later stage. The SPR/BLI assays on the one hand and chaotrope-based assays on the other hand measure different aspects of antibody avidity, with the former characterising the kinetics of antibody-antigen interactions in relation to time and the latter describing resistance of antibody-antigen binding to disruption by chaotropic reagents.

Surprisingly, it has been found by the present inventors that the in vitro determination of the avidity index of antibody-containing samples from vaccinated individuals at different time points after vaccination is an indicator of the avidity of the in vivo generated antibodies over time and therefore for the efficacy of the used virus vaccine. Suitable time points may include at least three different time points over a period of at least 180 days, preferably over at least one year after vaccination.

In a preferred embodiment the virus vaccine is a tetravalent dengue virus composition comprising four live, attenuated dengue virus strains. More preferably, the four live, attenuated dengue virus strains are:
(i) a chimeric dengue serotype 2/1 strain,
(ii) a dengue serotype 2 strain,
(iii) a chimeric dengue serotype 2/3 strain, and
(iv) a chimeric dengue serotype 2/4 strain.

In a further preferred embodiment, each one of the four live, attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 of the NS1 protein from glycine to asparagine, and in the NS3 gene at nucleotide 5270 from adenine to thymine resulting in an amino acid change at position 1725 of the NS3 protein from glutamine to valine.

The four live, attenuated Dengue virus strains may be TDV-1, TDV-2, TDV-3 and/or TDV-4. The nucleotide and amino acid sequence of TDV-1 is set forth in SEQ ID NO:1 and SEQ ID NO:2, respectively. The nucleotide and amino acid sequence of TDV-2 is set forth in SEQ ID NO:3 and SEQ ID NO:4, respectively. The nucleotide and amino acid sequence of TDV-3 is set forth in SEQ ID NO:5 and SEQ ID NO:6, respectively. The nucleotide and amino acid sequence of TDV-4 is set forth in SEQ ID NO:7 and SEQ ID NO:8, respectively.

In one embodiment, TDV-2 comprises in addition to the three attenuating mutations one or more mutations selected from:
a) a mutation in the prM gene at nucleotide 524 from adenine to thymine resulting in an amino acid change at position 143 from asparagine to valine, and/or
b) a silent mutation in the E gene at nucleotide 2055 from cytosine to thymine, and/or
c) a mutation in the NS2A gene at nucleotide 4018 from cytosine to thymine resulting in an amino acid change at position 1308 from leucine to phenylalanine, and/or
d) a silent mutation in the NS3 gene at nucleotide 5547 from thymine to cytosine, and/or
e) a mutation in the NS4A gene at nucleotide 6599 from guanine to cytosine resulting in an amino acid change at position 2168 from glycine to alanine, and/or
f) a silent mutation in the prM gene at nucleotide 900 from thymine to cytosine.

The silent mutation in the NS5 gene at nucleotide 8571 from cytosine to thymine of DEN-2 PDK-53 is not present in the TDV-2 strain.

In another embodiment, TDV-2 comprises in addition to the three attenuating mutations one or more mutations selected from:
g) a mutation in the prM gene at nucleotide 592 from adenine to guanine resulting in an amino acid change at position 166 from lysine to glutamine, and/or
h) a mutation in the NS5 gene at nucleotide 8803 from adenine to guanine resulting in an amino acid change at position 2903 from isoleucine to valine.

In one embodiment, TDV-1 comprises in addition to the three attenuating mutations one or more mutations selected from:
a) a mutation in the NS2A gene at nucleotide 4018 from cytosine to thymine resulting in an amino acid change at position 1308 from leucine to phenylalanine, and/or
b) a silent mutation in the NS3 gene at nucleotide 5547 from thymine to cytosine, and/or
c) a mutation in the NS4A gene at nucleotide 6599 from guanine to cytosine resulting in an amino acid change at position 2168 from glycine to alanine, and/or d) a silent mutation in the E gene at nucleotide 1575 from thymine to cytosine, and/or
e) a silent mutation in the junction site between the prM-E gene and the DEN-2 PDK-53 backbone at nucleotide 453 from adenine to guanine, and/or
f) a mutation in the junction site between the prM-E gene and the DEN-2 PDK-53 backbone at nucleotides 2381/2382 from thymine-guanine to cytosine-cytosine resulting in an amino acid change at position 762 from valine to alanine.

In another embodiment, TDV-1 comprises in addition to the three attenuating mutations one or more mutations selected from:
g) a mutation in the NS2A gene at nucleotide 3823 from adenine to cytosine resulting in an amino acid change at position 1243 from isoleucine to leucine, and/or
h) a mutation in the NS2B gene at nucleotide 4407 from adenine to thymine resulting in an amino acid change at position 1437 from glutamine to asparagine, and/or
i) a silent mutation in the NS4B gene at nucleotide 7311 from adenine to guanine.

In one embodiment, TDV-3 comprises in addition to the three attenuating mutations one or more mutations selected from:
a) a mutation in the NS2A gene at nucleotide 4012 from cytosine to thymine resulting in an amino acid change at position 1306 from leucine to phenylalanine, and/or
b) a silent mutation in the NS3 gene at nucleotide 5541 from thymine to cytosine, and/or
c) a mutation in the NS4A gene at nucleotide 6593 from guanine to cytosine resulting in an amino acid change at position 2166 from glycine to alanine, and/or
d) a silent mutation in the junction site between the prM-E gene and the DEN-2 PDK-53 backbone at nucleotide 453 from adenine to guanine, and/or
e) a mutation in the junction site between the prM-E gene and the DEN-2 PDK-53 backbone at nucleotides 2375/2376 from thymine-guanine to cytosine-cytosine resulting in an amino acid change at position 760 from valine to alanine, and/or
f) a silent mutation in the prM gene at nucleotide 552 from cytosine to thymine, and/or
g) a mutation in the E gene at nucleotide 1970 from adenine to thymine resulting in an amino acid change at position 625 from histidine to leucine.

In another embodiment, TDV-3 comprises in addition to the three attenuating mutations one or more mutations selected from:
h) a mutation in the E gene at nucleotide 1603 from adenine to thymine resulting in an amino acid change at position 503 from threonine to serine, and/or
i) a silent mutation in the NS5 gene at nucleotide 7620 from adenine to guanine.

In one embodiment, TDV-4 comprises in addition to the three attenuating mutations one or more mutations selected from:
a) a mutation in the NS2A gene at nucleotide 4018 from cytosine to thymine resulting in an amino acid change at position 1308 from leucine to phenylalanine, and/or
b) a silent mutation in the NS3 gene at nucleotide 5547 from thymine to cytosine, and/or
c) a mutation in the NS4A gene at nucleotide 6599 from guanine to cytosine resulting in an amino acid change at position 2168 from glycine to alanine, and/or
d) a silent mutation in the junction site between the prM-E gene and the DEN-2 PDK-53 backbone at nucleotide 453 from adenine to guanine, and/or
e) a mutation in the junction site between the prM-E gene and the DEN-2 PDK-53 backbone at nucleotides 2381/2382 from thymine-guanine to cytosine-cytosine resulting in an amino acid change at position 762 from valine to alanine, and/or
f) a mutation in the C gene at nucleotide 396 from adenine to cytosine resulting in an amino acid change at position 100 from arginine to serine, and/or
g) a silent mutation in the E gene at nucleotide 1401 from adenine to guanine, and/or
h) a mutation in the E gene at nucleotide 2027 from cytosine to thymine resulting in an amino acid change at position 644 from alanine to valine, and/or
i) a mutation in the E gene at nucleotide 2275 from adenine to cytosine resulting in an amino acid change at position 727 from methionine to leucine.

In another embodiment, TDV-4 comprises in addition to the three attenuating mutations one or more mutations selected from:
j) a silent mutation in the C gene at nucleotide 225 from adenine to thymine, and/or
k) a mutation in the NS2A gene at nucleotide 3674 from adenine to guanine resulting in an amino acid change at position 1193 from asparagine to glycine, and/or
l) a mutation in the NS2A gene at nucleotide 3773 from adenine to an adenine/guanine mix resulting in an amino acid change at position 1226 from lysine to a lysine/asparagine mix, and/or
m) a silent mutation in the NS3 gene at nucleotide 5391 from cytosine to thymine, and/or
aa) a mutation in the NS4A gene at nucleotide 6437 from cytosine to thymine resulting in an amino acid change at position 2114 from alanine to valine, and/or
bb) a silent mutation in the NS4B gene at nucleotide 7026 from thymine to a thymine/cytosine mix, and/or
cc) a silent mutation in the NS5 gene at nucleotide 9750 from adenine to cytosine.

In a further aspect a method of preparing a virus-like particle (VLP) attached to a biosensor suitable for SPR or BLI is provided, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

The VLPs and the pairs of binding molecules are as defined above. Preferably, the VLP is biotinylated and the biosensor has streptavidin attached to its surface. In a further preferred embodiment, the VLP is covalently linked to a biosensor having an amine-reactive surface.

The VLPs attached to the biosensors obtainable by the above methods are also encompassed by the present invention.

In a further aspect a method of preparing a live virus or an inactivated virus attached to a biosensor suitable for SPR or BLI is provided, wherein the method comprises attaching said live virus or said inactivated virus to the biosensor by hydrophobic interaction of said live virus or said inactivated virus with a capture reagent linked to the surface of the biosensor.

In a preferred embodiment said live virus or said inactivated virus is attached to the biosensor by hydrophobic interaction of said live virus or said inactivated virus with a capture reagent linked to the surface of the biosensor. More preferably, the capture reagent comprises aminopropylsilane.

The live virus or inactivated virus attached to the biosensors obtainable by the above methods are also encompassed by the present invention.

### EXAMPLES

### Example 1: Reactivity of Avidity Assay Using Antibodies from TDV-Vaccinated Healthy Individuals

Material and Methods

Sample and Reagents

8 vaccinated subjects with no anti-dengue virus-specific response before vaccination have been selected from the DEN-203 clinical trial (ClinicalTrials.gov identifier NCT01511250), i.e. a phase 2 clinical trial from Puerto Rico, Colombia, Singapore, and Thailand and sera from days 0, 28, 90, 120, 180 and 360 days post vaccination were obtained from these subjects. The sera were stored at −80° C. until use and thawed at 4° C. storage overnight before purification. Dengue Virus Like particles were purchased from Native antigen; Dengue 1 strain: Nauru/Western Pacific/1974 (UniProtKB/Swiss-Prot: P17763.2), Dengue 2 strain: Thailand/16681/84 (UniProtKB/Swiss-Prot: P29990.1), Dengue 3 strain: Sri Lanka/1266/2000 (UniProtKB/Swiss-Prot: Q6YMS4.1) and Dengue 4 strain: Dominica/814669/1981 (UniProtKB/Swiss-Prot: P09866.2). 20% of Envelope protein, E-protein, were replaced by the corresponding sequence of Japanese encephalitis strain SA-14 (UniProtKB/Swiss-Prot: P27395.1), amino acid sequence 397-495. SA-Biosensor, biosensor coated with Streptavidin, was purchased from Forte Bio.

Antibody Purification

IgG were purified from 200 μL of sera by Protein G Sepharose (GE). Briefly, 200 μL of sera were mixed with 3 mL Dulbecco's Phosphate-buffered saline (D-PBS) and 0.6 mL 50% Protein G Sepharose in a 15 mL centrifuge tube. These centrifuged tubes were mixed for 90 min at room temperature with a shaker. After centrifugation, Protein G Sepharose slurry was transferred to 24 well Unifilter (GE). The slurry was washed with D-PBS 4 times and eluted with 0.1M Glycine HCl pH2.7 for 4 times. The eluates were immediately neutralized to pH 7.0 to 7.5 with 1M Tris HCl pH8.0. The solution was buffer-exchanged with Amicon Ultra 4 (Millipore MWCO 30 KDa). The absorption at 280 nm in these antibody solutions was measured by NanoDrop 2000 (Thermo) and the IgG concentration was calculated. Then these samples were diluted with D-PBS to 2.5 mg/mL for each sample ID. The antibody purity was confirmed by SDS PAGE (NuPAGE 4-12% Bis-Tris Gel, Thermo). 2 μg protein sample was reduced at 70° C. for 10 min and applied to the gel. These gels were stained by simple stain blue (Thermo) and these images were captured by ChemoDoc Touch imaging system (Bio Rad) and calculated purity was confirmed more than 80% from both IgL and IgH band by Image Lab software (Bio-Rad).

Optimization of Biotinylation of Dengue VLP

Biotinylation of Dengue VLP was optimized using 20, 50 and 100 excess mole EZ-Link sulfo-NHS-Biotin. 80 μg of DENV1, 2, 3 and 4 VLPs were reacted for 60 min at room temperature respectively. After biotinylation, the excess biotinylation reagents were removed and the biotinylated VLPs were buffer-exchanged with D-PBS using Amicon Ultra 4 (Millipore MWCO 30 KDa). Biotinylation was evaluated by Octet Avidity assay using purified IgG from DEN203 sample 1053005 at 90 Days sera.

Preparation of Biotinylated Dengue VLP

For DEN203 avidity assays, 200 μg of Dengue Virus Like Particle, VLP, type 1, 2, 3 and 4 (Native Antigen) were biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 kDa). The biotinylated Dengue VLP solutions were adjusted to a concentration of 0.4 mg/mL and were stored at −80° C. until use.

Avidity Assay

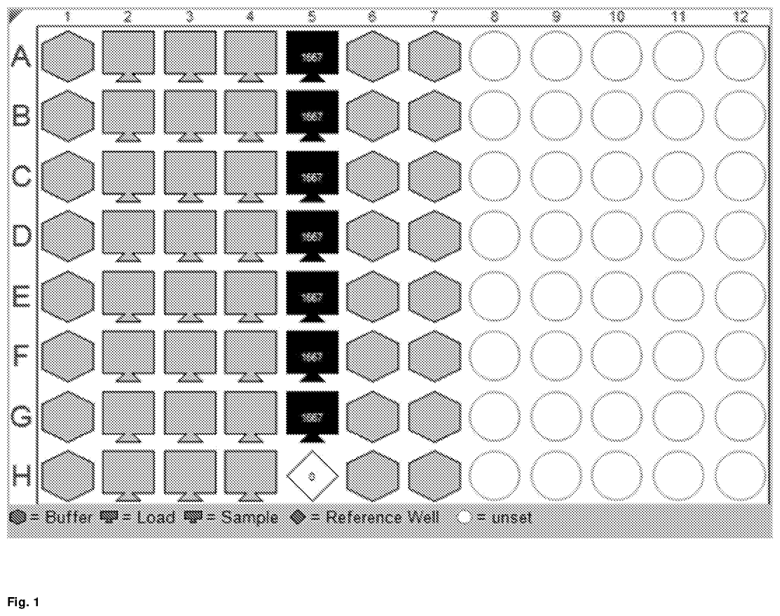

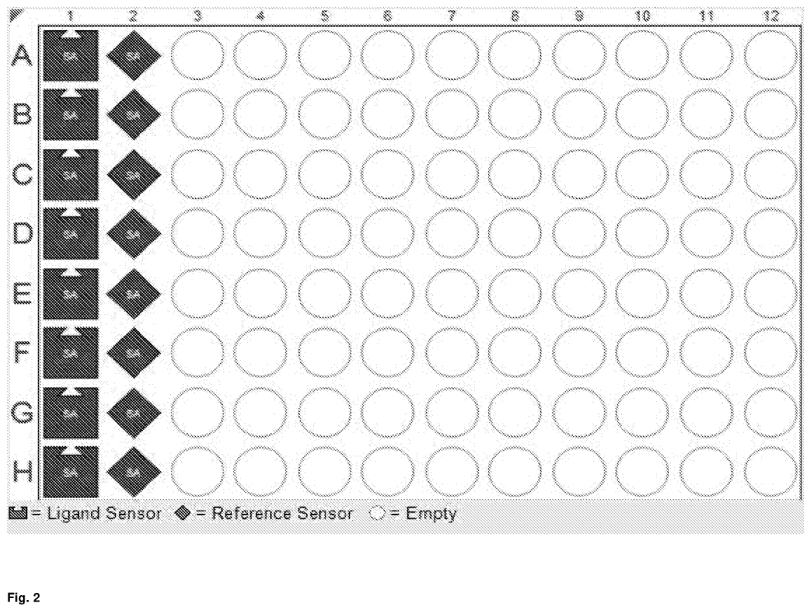

Avidity was measured by Octet 96 red (Forte Bio) using SA biosensor (Forte Bio). Briefly, SA biosensors were hydrated with D-PBS at least 10 min before an analysis. 5 ug/mL biotinylated Dengue VLP, in 0.1% BSA phosphate-buffered saline with Tween-20 (PBS-T) was captured on the SA biosensor and then the excess streptavidin on the surface was blocked with 50 μg/mL Biocytin (Thermo). 250 ug/mL anti-DENV polyclonal antibodies purified from DEN203 patients' sera in 0.1% BSA PBS-T were bound to the biosensor for 1800 sec and then the sensors were incubated in 0.1% BSA PBS-T 0.35M NaCl for 1200 sec to dissociate the binding antibody. These reactions were conducted at 30° C. and assay plates were shaken at 1000 rpm. (FIGS. 1 and 2, Tables 1 and 2).

TABLE 1
Example of plate layout

| Well | | | | Conc. |
|---|---|---|---|---|
| Location | Well Type | Sample ID | Description | ug/mL |
| A1-H1 | Buffer | 0.1% BSA PBST | | |
| A2-H2 | Load | Biotin DENV3 | 0.1% BSA PBST | |
| | | VLP 5 ug/mL | | |
| A3-H3 | Load | 0.1% BSA PBST | | |
| A4-H4 | Load | 50 ug/mL Biocytin | 0.1% BSA PBST | |
| A5 | Sample | 1053010 0 day | 0.1% BSA PBST | 250 |
| B5 | Sample | 1053010 28 day | 0.1% BSA PBST | 250 |
| C5 | Sample | 1053010 90 day | 0.1% BSA PBST | 250 |
| D5 | Sample | 1053010 120 day | 0.1% BSA PBST | 250 |
| E5 | Sample | 1053010 180 day | 0.1% BSA PBST | 250 |
| F5 | Sample | 1053010 360 day | 0.1% BSA PBST | 250 |
| G5 | Sample | PC sera IgG | 0.1% BSA PBST | 250 |
| | | 250 ug/mL | | |
| H5 | Reference | no AB | 0.1% BSA PBST | 0 |
| | Well | | | |
| A6-H6 | Buffer | 0.1% BSA PBST | | |
| A7-H7 | Buffer | 0.1% BSA PBST + | | |
| | | 0.35M NaCl | | |

TABLE 2
Assay conditions
Assay program

| | | | | | Assay |
|---|---|---|---|---|---|
| Assay Step | Step Data | Sensor | Sample | | Time |
| Number | Name | Column | Column | Step Type | sec |
| 1 | Sensor Check | 1 | 1 | Baseline | 30 |
| 2 | Loading | 1 | 2 | Loading | 600 |
| 3 | Blocking | 1 | 4 | Loading | 200 |
| | Biocytin | | | | |
| 4 | Baseline | 1 | 6 | Baseline | 180 |
| 5 | Association | 1 | 5 | Association | 1800 |
| | 1800 | | | | |
| 6 | Dissociation | 1 | 7 | Dissociation | 1200 |
| | 1200 | | | | |
| 7 | Sensor Check | 2 | 1 | Baseline | 30 |
| 8 | Loading | 2 | 3 | Loading | 600 |
| 9 | Blocking | 2 | 4 | Loading | 200 |
| | Biocytin | | | | |
| 10 | Baseline | 2 | 6 | Baseline | 180 |
| 11 | Association | 2 | 5 | Association | 1800 |
| | 1800 | | | | |
| 12 | Dissociation | 2 | 7 | Dissociation | 1200 |
| | 1200 | | | | |

For the negative subtractions, a double subtraction protocol was applied with a combination of antibody DENV VLP; antibody no DENV VLP; no antibody DENV VLP; and no antibody/no DENV VLP to assess the dissociation rate precisely. Data analysis was conducted by Octet Data Analysis Software (Forte Bio, version 9.0.0.10). For evaluation, two parameters for antibody avidity have been assessed. The response, correlated with the anti-DENV antibody concentration, was measured by the response values at 1800 sec association time. k_{off}, the antibody dissociation rate, showing strength of antibody binding, was measured by Langmuir 1:1 binding model fitting from 30 to 600 sec for dissociation. For some of the samples the dissociation rate was not measured due to too low dissociation. In these cases, dissociation rates were extrapolated to 2.0×10⁻⁵, detectable dissociation from 0 to 1200 sec for 5% signal decrease. Avidity index was calculated by the following equation: Avidity index=response/k_{off}. The assay was measured twice for each sample. Average values are shown.

Microneutralization Titer (MNT) Assay

Titers of antibodies neutralizing dengue live virus serotypes 1, 2, 3 and 4 were measured by the method reported by Osorio et al., Lancet Infect Dis 14 (2014), 830-838.

Statistics Analysis

All the data were analyzed with one-way ANOVA by Graph Pad Prizm (ver 7.02 GraphPad software). MNT titer and Avidity index values were converted to Log 10 value and then analyzed by Kruskal-Wallis one-way and ordinary one-way ANOVA, respectively. Response and k_{off} were analyzed by ordinary one-way ANOVA.

Results

Antibody Purification

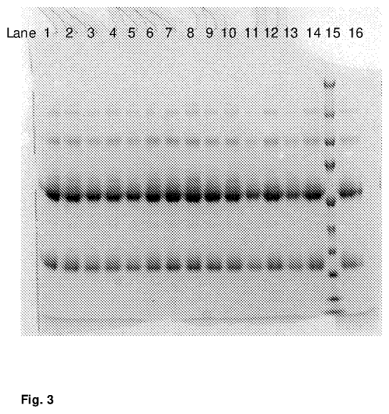

Forty-eight IgG samples were purified from DEN203 human sera and showed more than 88% purity for each sample. The antibody solution was stored at 4° C. until the assay. The yield of IgG from sera sample were from 4.8 mg/mL to 19.0 mg/mL. FIG. 3 shows the SDS PAGE analysis of anti-DENV Ab purified from DEN203 sera sample. The distribution of the samples in the lanes of the gel is shown in Table 3.

TABLE 3

| Lane | subject ID | days | purity % |
|---|---|---|---|
| 1 | 1053009 | 0 | 91.4 |
| 2 | 1053012 | 28 | 90.5 |
| 3 | 1053009 | 28 | 93.8 |
| 4 | 1053012 | 90 | 91.7 |
| 5 | 1053009 | 90 | 93.4 |
| 6 | 1053012 | 120 | 91.7 |
| 7 | 1053009 | 120 | 91.3 |
| 8 | 1053012 | 180 | 91.6 |
| 9 | 1053009 | 180 | 92.2 |
| 10 | 1053012 | 360 | 91.4 |
| 11 | 1053009 | 360 | 94.6 |
| 12 | 1063001 | 0 | 88.8 |
| 13 | 1053012 | 0 | 92.4 |
| 14 | 1063001 | 28 | 89.7 |
| 16 | 1063001 | 90 | 89.1 |

Optimization of Biotinylation of Dengue VLPs

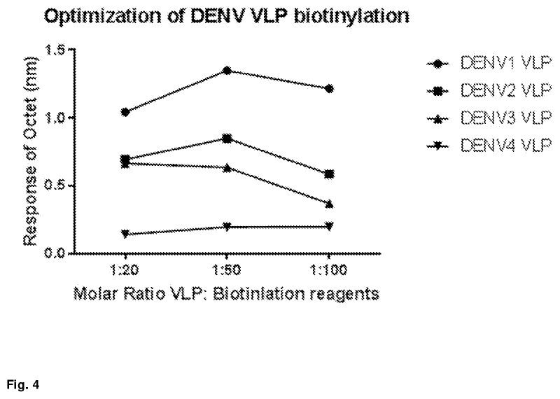

We varied the Dengue VLP/biotinylated reagents ratio from 1:20, 1:50 to 1:100 excess molar. Responses, i.e. binding of anti-Dengue antibody, were increased when biotinylated from 20 to 50 excess moles. However, if 100 excess moles were used, the response, binding of anti-Dengue antibodies, was decreased. From these optimizations, the 50 fold excess mole of biotinylation reagents was chosen for 60 min incubations (FIG. 4).

Avidity Assay

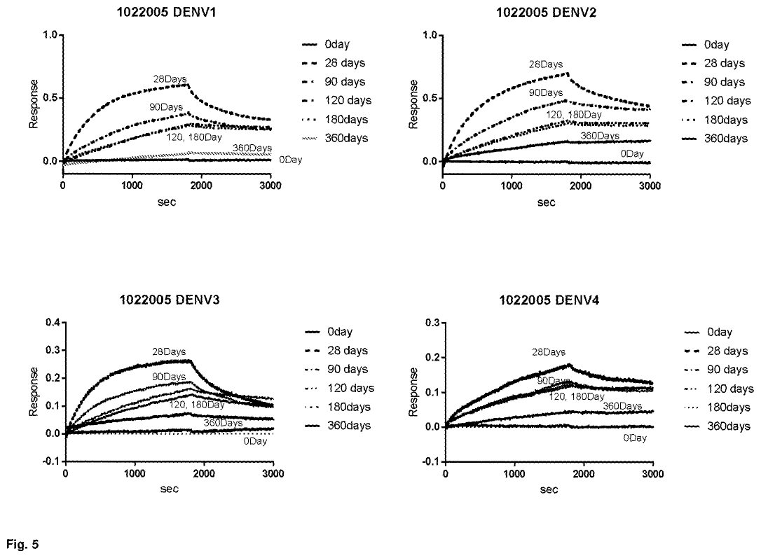

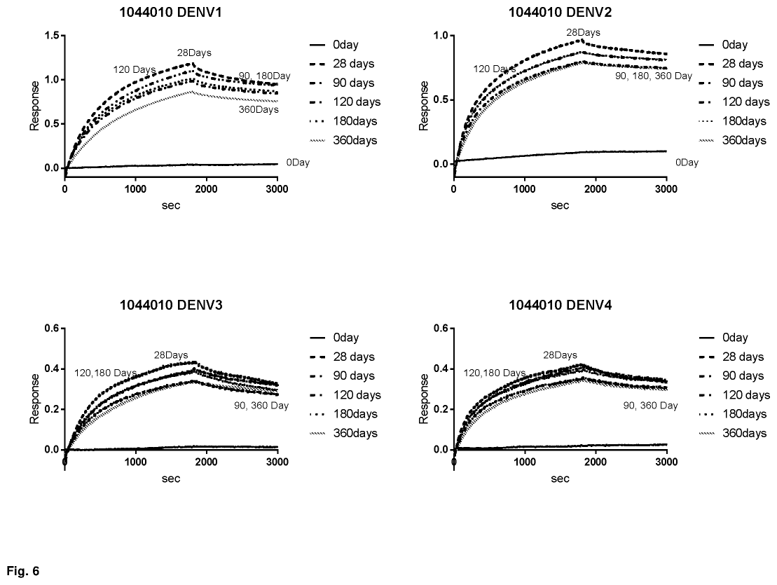

Avidity assays were conducted for samples from eight subjects which were dengue-naïve before vaccination from day 0, 28, 90, 120, 180 and 360 days post vaccination. Hence, 48 samples for each of the four serotypes of Dengue VLP were measured. Two biosensor image patterns that represent featured patterns were shown in FIGS. 5 and 6, respectively.

Subject ID 1022005 showed a strong affinity maturation process. The response to all dengue serotypes was increased at day 28 after immunization, but binding antibodies dissociated easily (Day 28 to DENV2; Response: 0.694 nm, k_{off}: 4.06E−04 1/s and Avidity index: 1721 nm*s; FIG. 5). At day 90, the binding of the antibodies was decreased, but their dissociation from dengue VLP was reduced (Day 90 to DENV2; Response: 0.311 nm, k_{off}: 7.93E−05 1/s and Avidity index: 6524 nm*s; FIG. 5). From that point of view, a clear affinity maturation was observed.

On the other hand, subject ID 1044010 showed high response and small dissociation rate even at day 28. (Day 28 to DENV2; Response: 1.003 nm, k_{off}: 7.65E−05 1/s and Avidity index: 13920 nm*s; FIG. 6) and kept its antibody strength until day 360 (Day 360 to DENV2; Response; 0.782 nm, k_{off}; 4.04E−05 1/s Avidity index; 25920 nm*s; FIG. 6). These data suggest that the vaccination with the dengue vaccine was effective even one year after the immunization. Eight subjects were compared from day 0 to day 360 after vaccination. A summary of the avidity data from these eight subjects is shown in Table 5.

TABLE 5
Summary of Avidity Assay of DEN203
DENV1

| | | MNT | | | response | | | Koff | | | Avidity index | | |
|---|---|---|---|---|---|---|---|---|---|---|---|---|---|
| days | N | GEM | | SEM | MEAN | | SEM | MEAN | | SEM | GEM | | SEM |
| 0 | 8 | 5 | ± | 1 | 0.009 | ± | 0.006 | ND | ± | ND | 0 | ± | 0 |
| 28 | 8 | 364 | ± | 172 | 0.728 | ± | 0.082 | 2.65E−04 | ± | 5.93E−05 | 3065 | ± | 677 |
| 90 | 8 | 207 | ± | 65 | 0.657 | ± | 0.067 | 2.22E−04 | ± | 4.23E−05 | 3238 | ± | 790 |
| 120 | 8 | 293 | ± | 62 | 0.739 | ± | 0.078 | 1.64E−04 | ± | 2.38E−05 | 4632 | ± | 571 |
| 180 | 8 | 190 | ± | 66 | 0.628 | ± | 0.082 | 1.60E−04 | ± | 2.01E−05 | 3924 | ± | 647 |
| 360 | 8 | 175 | ± | 317 | 0.549 | ± | 0.151 | 1.37E−04 | ± | 2.48E−05 | 3741 | ± | 997 |

DENV2

| | | MNT | | | response | | | Koff | | | Avidity index | | |
|---|---|---|---|---|---|---|---|---|---|---|---|---|---|
| Days | N | GEM | | SEM | MEAN | | SEM | MEAN | | SEM | GEM | | SEM |
| 0 | 8 | 5 | ± | 0 | 0.012 | ± | 0.008 | ND | ± | ND | 0 | ± | 0 |
| 28 | 8 | 1396 | ± | 2583 | 0.684 | ± | 0.055 | 1.77E−04 | ± | 4.26E−05 | 4648 | ± | 1474 |
| 90 | 8 | 538 | ± | 294 | 0.634 | ± | 0.041 | 1.37E−04 | ± | 2.73E−05 | 5737 | ± | 2159 |
| 120 | 8 | 562 | ± | 291 | 0.661 | ± | 0.056 | 1.02E−04 | ± | 1.56E−05 | 7473 | ± | 1926 |
| 180 | 8 | 453 | ± | 301 | 0.590 | ± | 0.063 | 9.02E−05 | ± | 1.45E−05 | 7207 | ± | 2054 |
| 360 | 8 | 353 | ± | 133 | 0.531 | ± | 0.115 | 6.75E−05 | ± | 1.32E−05 | 9306 | ± | 2992 |

DENV3

| | | MNT | | | response | | | Koff | | | Avidity index | | |
|---|---|---|---|---|---|---|---|---|---|---|---|---|---|
| Days | N | GEM | | SEM | MEAN | | SEM | MEAN | | SEM | GEM | | SEM |
| 0 | 8 | 5 | ± | 0 | −0.001 | ± | 0.005 | ND | ± | ND | 0 | ± | 0 |
| 28 | 8 | 174 | ± | 144 | 0.291 | ± | 0.030 | 3.65E−04 | ± | 9.87E−05 | 986 | ± | 225 |
| 90 | 8 | 160 | ± | 151 | 0.281 | ± | 0.029 | 2.76E−04 | ± | 6.93E−05 | 1246 | ± | 386 |
| 120 | 8 | 247 | ± | 60 | 0.312 | ± | 0.029 | 2.10E−04 | ± | 1.83E−05 | 1540 | ± | 284 |
| 180 | 8 | 199 | ± | 300 | 0.267 | ± | 0.033 | 2.14E−04 | ± | 3.26E−05 | 1584 | ± | 600 |
| 360 | 8 | 200 | ± | 645 | 0.225 | ± | 0.050 | 1.98E−04 | ± | 2.33E−05 | 1239 | ± | 494 |

DENV4

| | | MNT | | | response | | | Koff | | | Avidity index | | |
|---|---|---|---|---|---|---|---|---|---|---|---|---|---|
| days | N | GEM | | SEM | MEAN | | SEM | MEAN | | SEM | GEM | | SEM |
| 0 | 8 | 5 | ± | 0 | 0.004 | ± | 0.004 | ND | ± | ND | 0 | ± | 0 |
| 28 | 8 | 62 | ± | 45 | 0.257 | ± | 0.035 | 2.42E−04 | ± | 4.15E−05 | 1145 | ± | 299 |
| 90 | 8 | 26 | ± | 11 | 0.224 | ± | 0.028 | 2.06E−04 | ± | 1.45E−05 | 1059 | ± | 193 |
| 120 | 8 | 48 | ± | 21 | 0.260 | ± | 0.031 | 1.57E−04 | ± | 1.84E−05 | 1894 | ± | 498 |
| 180 | 8 | 40 | ± | 18 | 0.227 | ± | 0.035 | 1.70E−04 | ± | 1.41E−05 | 1504 | ± | 235 |
| 360 | 8 | 34 | ± | 39 | 0.196 | ± | 0.047 | 1.17E−04 | ± | 2.63E−05 | 2200 | ± | 773 |

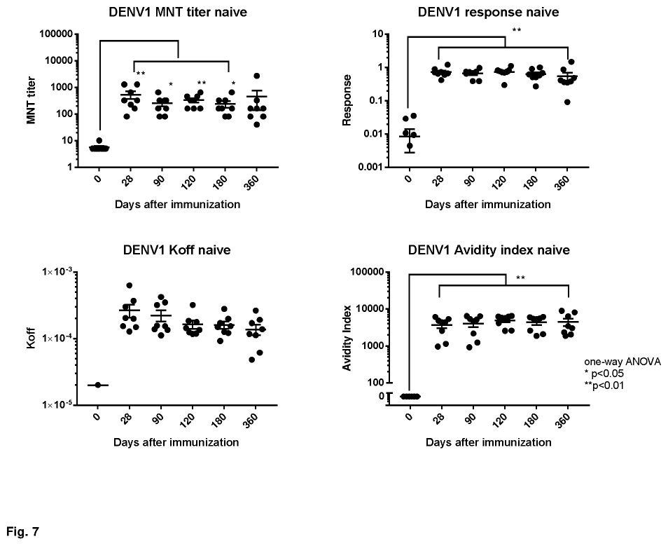

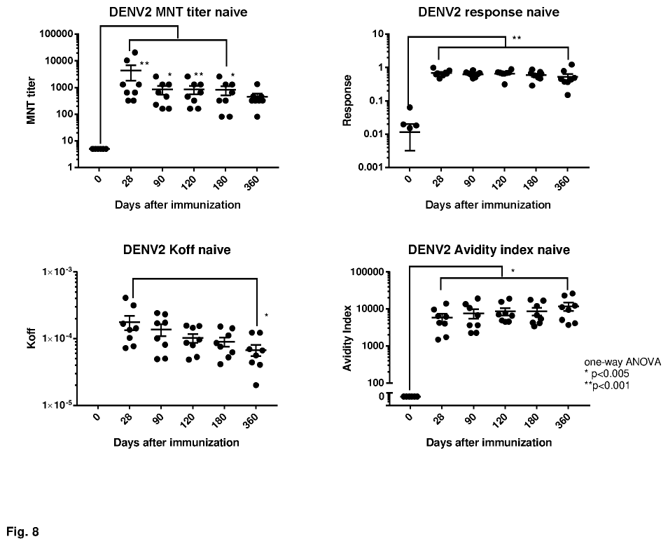

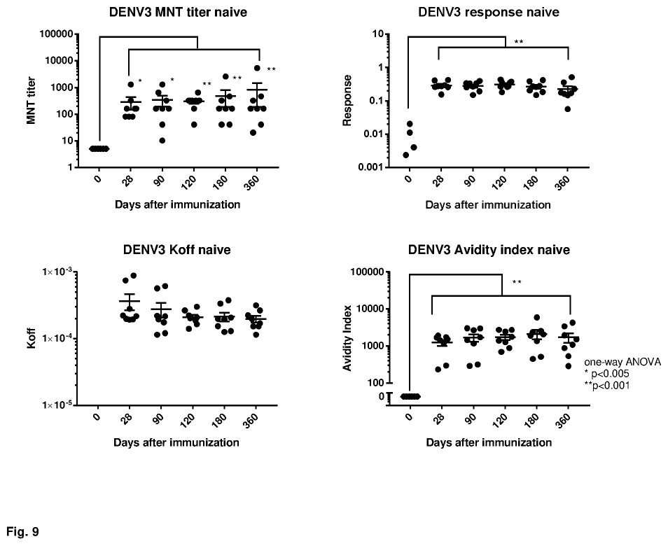

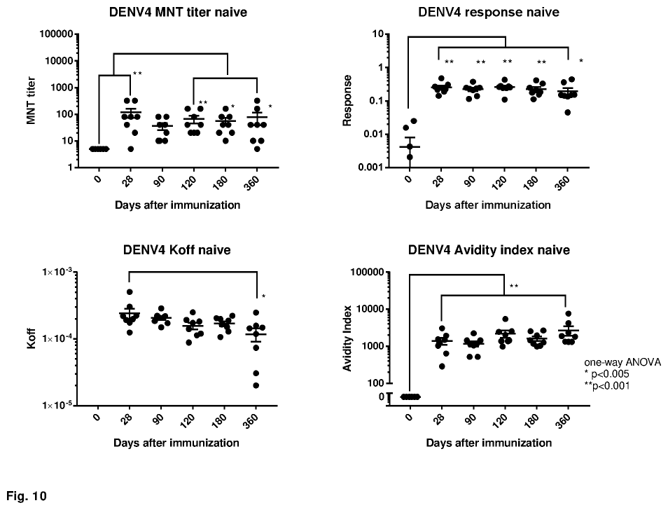

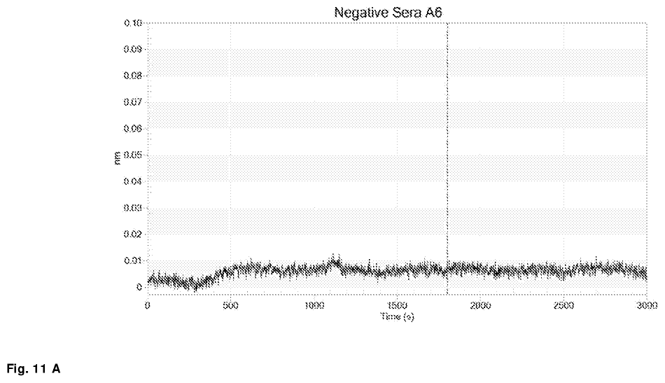

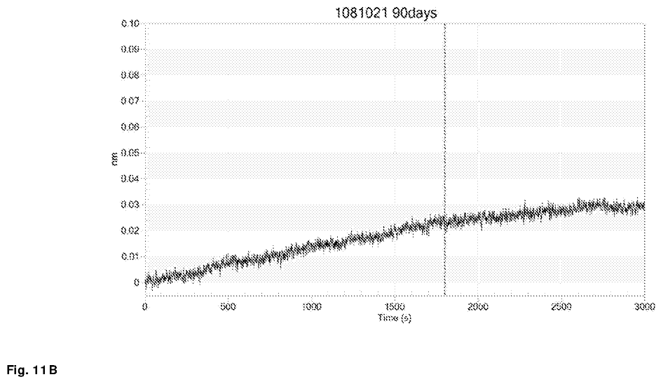

For dengue serotype 2, FIG. 8, MNT titer was increased at day 28 and then gradually decreased. However, the response that reflects anti Dengue 2 IgG content, was increased at day 28, but some of the subjects kept the response even at day 360. There were significant differences between day 0 and day 28 to day 360 (one-way ANOVA p<0.01). Antibody dissociation rate, k_{off}, was decreased from day 28 to day 360, there were significant differences between day 28 and day 360 (by one-way ANOVA p<0.05). These findings showed that the strength of anti-Dengue antibodies kept in patients for over one year was increased over time. The avidity index, showing strength of antibody binding was still at a high level at day 360. There were also significant differences between day 0 and day 28 to day 360 (by one-way ANOVA p<0.05). There were almost the same responses towards all serotypes, DENV1, 3 and 4 respectively, FIGS. 7, 9 and 10. These findings suggest that immunization with the dengue vaccine sustains immune responses and these high antibody avidities may protect from infections by dengue virus.

### Example 2: Reactivity and Specificity of Avidity Assay Using Anti-DENV Antibody Panels (VLP/AR2G, VLP/SA Biosensor)

Material and Methods

Materials

Dengue serotype-1, 2 3 and 4 were purchased from Native antigen and SA biosensor, AR2G biosensor and The Amine Reactive 2nd Generation (AR2G) Reagent Kit were purchased from Forte Bio, EZ-Link Sulfor-NHS_Biotin were purchased from Thermo Scientific. Anti-Dengue antibodies were purchased or prepared based on amino acid sequences or hybridomas. All, B7, C10, 2C8, 4G2, DV1-106, 2D22, 5J7, DV4-75, DV3-E60, WNV E60 DV1-106, 1M7 shown in Table 6.

TABLE 6
anti DENV monoclonal antibody panels

| Clones | Epitope Specificity | Reference |
|---|---|---|
| A11 | EDE Cross reactive | Dejnirattisai, W. Wongwiwat, S. Supasa, X. Zhang, X. |
| B7 | | Dai, A. Rouvinsky, A. Jumnainsong, C. Edwards, N. T. |
| C10 | | Quyen, T. Duangchinda, J. M. Grimes, W. Y. Tsai, C. Y. |
| 2C8 | | Lai, W. K. Wang, P. Malasit, J. Farrar, C. P. Simmons, Z. H. |
| | | Zhou, F. A. Rey, J. Mongkolsapaya, G. R. Screaton |
| | | A new class of highly potent, broadly neutralizing |
| | | antibodies isolated from viremic patients infected with |
| | | dengue virus |
| | | Nat. Immunol., 16 (2015), pp. 170-177 |
| 4G2 | Fusion Loop | Halstead S B, Venkateshan C N, Gentry M K, Larsen L K. |
| | Cross reactive | Heterogeneity of infection enhancement of dengue 2 |
| | | strains by monoclonal antibodies. |
| | | J Immunol. 1984 March; 132(3): 1529-32 |
| DV- | QE DENV1 Specific | Shrestha B, Brien J D, Sukupolvi-Petty S, Austin S K, |
| 106 | | Edeling M A, Kim T, O'Brien K M, Nelson C A, Johnson S, |
| | | Fremont D H, Diamond M S. |
| | | The development of therapeutic antibodies that neutralize |
| | | homologous and heterologous genotypes of dengue virus |
| | | type 1. |
| | | PLoS Pathog. 2010 Apr. 1; 6(4): e1000823 |
| 2D22 | QE DENV2 specific | R. de Alwis, S. A. Smith, N. P. Olivarez, W. B. Messer, J. P. |
| 5J7 | | Huynh, W. M. Wahala, L. J. White, M. S. Diamond, R. S. |
| | | Baric, J. E. Crowe Jr., A. M. de Silva |
| | | Identification of human neutralizing antibodies that bind to |
| | | complex epitopes on dengue Virions |
| | | Proc. Natl. Acad. Sci. U.S.A., 109 (2012), pp. 7439-7444 |
| WNV- | Fusion loop | Oliphant T, Engle M, Nybakken G E, Doane C, Johnson S, |
| E60 | Cross reactive | Huang L, Gorlatov S, Mehlhop E, Marri A, Chung K M, |
| | | Ebel G D, Kramer L D, Fremont D H, Diamond M S. |
| | | Development of a humanized monoclonal antibody with |
| | | therapeutic potential against West Nile virus. |
| | | Nat Med. 2005 May; 11(5): 522-30 |
| DV3- | QE DENV3 specific | Brien J D, Austin S K, Sukupolvi-Petty S, O'Brien K M, |
| E60 | | Johnson S, Fremont D H, Diamond M S, |
| | | Genotype-specific neutralization and protection by |
| | | antibodies against dengue virus type 3 |
| | | J Virol. 2010 October; 84(20): 10630-43 |
| DV4- | QE DENV4 specific | Sukupolvi-Petty S, Brien J D, Austin S K, Shrestha B, |
| 75 | | Swayne S, Kahle K, Doranz B J, Johnson S, Pierson T C, |
| | | Fremont D H, Diamond M S. |
| | | Functional analysis of antibodies against dengue virus type |
| | | 4 reveals strain-dependent epitope exposure that impacts |
| | | neutralization and protection. |
| | | J Virol. 2013 August; 87(16): 8826-42 |
| 1M7 | Fusion loop | Smith S A, de Alwis A R, Kose N, Harris E, Ibarra K D, |
| | Cross reactive | Kahle K M, Pfaff J M, Xiang X, Doranz B J, de Silva A M, |
| | | Austin S K, Sukupolvi-Petty S, Diamond M S, Crowe J E Jr. |
| | | The potent and broadly neutralizing human dengue virus- |
| | | specific monoclonal antibody 1C19 reveals a unique cross- |
| | | reactive epitope on the be loop of domain II of the |
| | | envelope protein. |
| | | MBio. 2013 Nov. 19; 4(6): e00873-13 |

Evaluation of Dengue VLP Coupled to AR2G Biosensor Using Anti-Dengue Antibody Panel

This assay was measured by Octet Red (Forte Bio). Coupling Dengue VLP to AR2G biosensor following the instructions of the Amine Reactive 2^{nd} Generation (AR2G) reagent kit. First, AR2G biosensor was hydrated with AR2G in PBS for 5 min before the reaction. AR2G biosensor was activated with 20 mM EDC (1-Ethyl-3-[3-dimethylaminopropyl] carbodiimide hydrochloride) and 10 mM S-NHS (N-hydroxysulfosuccinimide) for 300 sec. The activated biosensor was reacted with 10 ug/mL Dengue VLP-1, -2, -3 and -4 in 10 mM Acetate pH 6 buffer for 600 sec respectively. The VLP coupled biosensor was quenched with 1M ethanolamine pH 8.5 for 300 sec. All reactions were done at 1000 rpm plate shaking at 30° C. After the coupling of Dengue VLP to the biosensor, binding to the anti-Dengue antibody panels was confirmed. The antibody solution was diluted to 10 ug/mL in 0.1% BSA PBST. These antibodies were associated to Dengue VLP for 900 sec and these antibodies were dissociated in the same buffer for 1800 sec. This assay was conducted at 30° C. with 1000 rpm shaking plate. All solution volume was 200 uL in 96 well black plate (Greiner Bio).

Biotinylation of Dengue VLP

200 ug of Dengue Virus Like Particle (VLP), type 1, 2, 3 and 4 (Native Antigen) was biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 KDa).

Evaluation of Biotinylated Dengue VLP/SA Biosensor Using Anti-Dengue Antibody Panel

This assay was measured by Octet Red (Forte Bio). SA biosensor was hydrated with PBS for 5 min before the assay. Biotinylated Dengue VLP was diluted to 5 ug/mL in 0.1% BSA PBST and bound to SA Biosensor for 600 sec. then binding to the anti-Dengue antibody panels was confirmed. The antibody solution was diluted to 10 ug/mL in 0.1% BSA PBST. These antibodies were associated to Dengue VLP for 600 sec and these antibodies were dissociated in the same buffer for 900 sec. This assay was conducted at 30° C. with 1000 rpm shaking plate. All solution volume was 200 uL in 96 well black plate (Greiner Bio).

Results

The results are shown in the following Table.

TABLE 7
Reactivity of anti-Dengue antibodies
panels to AR2G Dengue VLPs

| Clones | Specificity | Epitope | DENV1 | DENV2 | DENV3 | DENV4 |
|---|---|---|---|---|---|---|
| 4G2 | CR | Fusion | +++ | ++ | +++ | +++ |
| | | Loop | | | | |
| WNV-E60 | CR | Fusion | ++ | ++ | +++ | ++ |
| | | Loop | | | | |
| 2D22 | DENV2 | QE | − | ++ | − | − |
| DV3 E60 | DENV3 | QE | − | − | ++ | − |
| DV4 75 | DENV4 | QE | − | − | + | +++ |

−; no binding
+; weak binding
++; binding
+++; Strong binding
EDE; Envelope Dimer Epitope,
QE; Quarternary E-protein,
CR; Cross reactive,
VLP; Virus like particle

The cross-reactive antibodies 4G2 and WNV-E60 bound to each of DENV1 to DENV4 attached to the AR2G biosensor. The serotype-specific antibodies 2D22, DV3E60 and DV475 only bound to its serotype-specific DENV attached to the AR2G biosensor but did not bind to VLPs from other Dengue serotypes.

TABLE 8
Reactivity of anti-Dengue antibodies panels
to SA biosensor to biotinylated Dengue VLPs

| clones | epitope | specificity | DENV1 | DENV2 | DENV3 | DENV4 |
|---|---|---|---|---|---|---|
| 4G2 | Fusion | CR | +++ | +++ | ++ | ++ |
| | Loop | | | | | |
| WNV-E60 | Fusion | CR | ++ | +++ | ++ | + |
| | Loop | | | | | |
| 1M7 | Fusion | CR | +++ | +++ | +++ | ++ |
| | Loop | | | | | |
| DV1-106 | QE | DEN1 | + | − | − | − |
| 2D22 | QE | DEN2 | − | + | − | − |
| 5J7 | QE | DEN3 | − | − | +++ | − |
| DV4-75 | QE | DEN4 | − | − | − | +++ |

−; no binding
+; weak binding
++; binding
+++; Strong binding
EDE; Envelope Dimer Epitope,
QE; Quarternary E-protein,
CR; Cross reactive,
VLP; Virus like particle

These results demonstrate the suitability of AR2G coupling VLP strategies and biotinylated VLP strategies for avidity assays.

### Example 3: Propagation of Live Dengue 3 and 4 Virus

Materials and Methods

Materials

Dengue Wildtype strain type 16562 for Dengue 3 and wildtype strain type 1036 for Dengue 4 were used. Vero cells were obtained from WHO. Coring cell stacker 10 layer were purchased form Coring. For cell culture medium, Fetal bovine Serum, FBS, was obtained from Sigma Aldrich and DMEM 1× and Penicillin/Streptomycin solution were purchased from Gibco. For concentration, Viva Flow MWCO 100 kDa system were purchased from Sartorius. 60% sucrose solution and TNE buffer, Tris 10 mM, EDTA 1 mM, NaCl 100 mM pH8.0 were obtained from Teknova.

Preparation of Vero Cells

Vero cells were cultured in 10-layer hyperflask cell culture vessel with 10% FBS, DMEM and Pen/Strep Medium and confluent to vessels prior to the transfection.

Infection and Purification of Dengue Virus

Wildtype DENV strains, type 16562 for Dengue 3 and type 1036 for Dengue 4, was propagated in a 10-layer hyperflask cell culture vessel at a MOI of 0.01 using Vero cells for an incubation period of 9 days. Supernatants were collected at days 5, 7 and 9 post-infection and cells replenished with fresh virus growth media after each collection time point. Each collection day supernatant was clarified and filtered using Millipore 0.22 um filters to remove host cell debris. The clarified and filtered supernatants were subjected to tangential flow filtration, TFF Viva Flow MWCO 100 kDa, to concentrate virus stock and concentrated solution were overlaid with 20% Sucrose and centrifuged for 3 hours at 112,398×g (25,000 RPM), 4° C. to form the pellets. The formed virus pellets were resuspended in TNE buffer and frozen down at −80° C. The concentration of the viruses was estimated using BCA protein assay kit (Themo) for BSA as a standard.

SDS-PAGE

The purity of Dengue virus was confirmed by SDS PAGE (NuPAGE 4-12% Bis-Tris Gel, Thermo) 2 ug protein sample were reduced at 70° C. for 10 min and applied to the gel. These gels were stained by simple stain blue (Thermo) and these images were captured by ChemoDoc Touch imaging system (Bio-Rad).

Results

Live dengue virus was obtained from the propagation process with Vero cells. These purified viruses showed E protein (MW 55 kDa) and prM protein (MW 18 KDa) in SDS-PAGE.

### Example 4: Reactivity and Specificity of Avidity Assay Using Anti-DENV Antibody Panels

Materials

Dengue VLPs for serotype-3 and 4 were purchased from Native antigen and Live Dengue virus were propagated and purified as described in Example 5. SA biosensor, APS biosensor and Protein G biosensor were purchased from Forte Bio, EZ-Link Sulfor-NHS_Biotin were purchased from Thermo Scientific. Anti-Dengue antibodies were purchased or prepared based on amino acid sequences or hybridomas. All, B7, C10, 2C8, 4G2, 2D22, DV4-75, DV3-E60, WNV E60 DV1-106, 5J7 and 1M7 are shown in Table 6.

Biotinylation of Dengue VLP

200 ug of Dengue Virus Like Particle, VLP, type 3 and 4 (Native Antigen) were biotinylated with 50 excess moles of EZ-Link Sulfo-NHS-Biotin (Thermo) following the manufacturer's protocol. The reaction took place at room temperature for 60 min, after the reaction, the excess biotinylation reagents were removed and these biotinylated VLPs buffer-exchanged with D-PBS using Amicon Ultra 4 (Millipore MWCO 30 kDa).

Biotinylation of Anti-Dengue Monoclonal Antibody 4G2

50 ug of anti-Dengue antibody, 4G2, cross-reactive antibody to all serotypes, was biotinylated with 50 excess moles of EZ-Link Sulfo-NHS-Biotin (Thermo) following the manufacturer's protocol. The reaction was carried out at room temperature for 60 min. after the reaction, the excess biotinylation reagents were removed and these biotinylated VLPs buffer-exchanged with D-PBS using Amicon Ultra 4 (Millipore MWCO 30 kDa).

Evaluation of Live Virus and VLP Binding to Anti-Dengue Antibody/Protein G Biosensor

This assay was measured by Octet Red (Forte Bio). Protein G biosensor was hydrated with PBS for 5 min before the assay. The biosensor was dipped into 1 μg/mL of twelve anti-Dengue antibodies in 0.1% PBS and PBST for 600 sec. Then binding to Dengue live virus or VLP was confirmed. Live virus and VLP were diluted to 5 ug/mL in 0.1% BSA PBST. These solutions were associated to anti-Dengue antibody panel/Protein A biosensor for 900 sec and dissociated in the same buffer for 1800 sec. This assay was conducted at 30° C. with 1000 rpm shaking plate. All solution volume was 200 uL in 96 well black plate (Greiner Bio).

Evaluation of Anti-Dengue Antibody Panels Binding to Live Virus to APS Biosensor

This assay was measured by Octet Red (Forte Bio). APS biosensor was hydrated with PBS for 5 min before the assay. Dengue VLP or Live virus were diluted to 3 ug/mL in PBS and bound to APS Biosensor for 600 sec. Then the sensor was blocked with 1% BSA PBS for 300 sec. The binding to twelve anti-Dengue antibody panels was confirmed. The antibody solution was diluted to 10 ug/mL in 1% BSA PBS. These antibodies were associated to Dengue VLP or Live virus for 900 sec and these antibodies were dissociated in the same buffer for 1800 sec. This assay was conducted at 30° C. with 1000 rpm shaking plate. All solution volume was 200 uL in 96 well black plate (Greiner Bio).

Evaluation of Anti-Dengue Antibody Panels Binding to Biotinylated VLP to SA Biosensor.

This assay was measured by Octet Red (Forte Bio). SA biosensor was hydrated with PBS for 5 min before the assay. Biotinylated Dengue VLP was diluted to 5 ug/mL in 0.1% BSA PBST and bound to SA Biosensor for 600 sec. Then binding to twelve anti-Dengue antibody panels was confirmed. The antibody solution was diluted to 10 ug/mL in 0.1% BSA PBST. These antibodies were associated to Dengue VLP for 900 sec and these antibodies were dissociated in the same buffer for 1800 sec. This assay was conducted at 30° C. with 1000 rpm shaking plate. All solution volume was 200 uL in 96 well black plate (Greiner Bio).

Results

We confirmed Dengue live virus and VLP binding to anti-Dengue antibody panel.

TABLE 9
Reactivities of anti-Dengue antibody panels
to various avidity assay format of DENV3

| | | | | | Biotinylated |
|---|---|---|---|---|---|
| | | | APS LV/VLP capture | | VLP |
| Clone | Epitope | specificity | Live Virus | VLP | VLP |
| 1M7 | Fusion | CR | + | +++ | +++ |
| | Loop | | | | |
| WNV-E60 | Fusion | CR | + | ++ | ++ |
| | Loop | | | | |
| DV1-106 | QE | DEN1 | − | − | − |
| 2D22 | QE | DEN2 | + | − | − |
| 5J7 | QE | DEN3 | − | +++ | +++ |
| DV3-E60 | QE | DEN3 | ++ | + | − |
| DV4-75 | QE | DEN4 | − | − | − |

−; no binding
+; weak binding
++; binding
+++; Strong binding
EDE; Envelope Dimer Epitope,
QE; Quarternary E-protein,
CR; Cross reactive,
VLP; Virus like particle

TABLE 10
Reactivities of anti-Dengue antibody panels
to various avidity assay format of DENV4

| | | | | | Biotinylated |
|---|---|---|---|---|---|
| | | | APS LV/VLP capture | | VLP |
| clone | epitope | specificity | Live Virus | VLP | VLP |
| 1M7 | Fusion | CR | + | ++ | ++ |
| | Loop | | | | |
| DV1-106 | QE | DEN1 | − | − | − |
| 2D22 | QE | DEN2 | − | − | − |
| 5J7 | QE | DEN3 | − | − | − |
| DV3-E60 | QE | DEN3 | − | − | − |
| DV4-75 | QE | DEN4 | + | ++ | +++ |

−; no binding
+; weak binding
++; binding
+++; Strong binding
EDE; Envelope Dimer Epitope,
QE; Quarternary E-protein,
CR; Cross reactive,
VLP; Virus like particle

When IgG was captured to Protein G biosensor, these cross-reactive antibody panels and Dengue 3 or 4 specific antibodies reacted with both live virus.

### Example 5: Avidity Assay Using Live Virus

Materials and methods

Materials

Live Dengue virus serotype 3 were propagated and purified as outlined above. APS biosensor was purchased from Forte Bio. Dengue positive control sera and negative control sera were obtained from NIH. Sera samples were selected from DEN-203, phase 2 clinical trial from Puerto Rico, Colombia, Singapore, and Thailand. The sera were stored at −80° C. until use. These sera samples were thawed at 4° C. storage shelf overnight before purification.

Antibody Purification

IgG were purified from these 200 uL sera by Protein G Sepharose (GE). Briefly, 200 uL of sera were mixed with 3 mL D-PBS and 0.6 mL 50% Protein G Sepharose in 15 mL centrifuge tube. These centrifuged tubes were mixed for 90 min at room temperature with shaker. After centrifuging, Protein G Sepharose slurry were transferred to 24 well Unifilter (GE) and the gel was washed with D-PBS for 4 times and eluted with 0.1M Glycine HCl pH2.7 for 4 times. The eluates were immediately neutralized to pH 7.0 to 7.5 with 1M Tris HCl pH8.0. the solution was buffer-exchanged to Amicon Ultra 4 (Millipore MWCO 30 KDa). These antibody solutions are measured at A280 by NanoDrop 2000 (Thermo) and the IgG concentration was calculated. Then these samples were diluted with D-PBS to 2.5 mg/mL for each sample ID. The antibody purity was confirmed by SDS PAGE (NuPAGE 4-12% Bis-Tris Gel, Thermo). 2 ug protein sample were reduced at 70° C. for 10 min and applied to the gel. These gels were stained by simple stain blue (Thermo) and these images were captured by ChemoDoc Touch imaging system (Bio-Rad) and calculated purity was confirmed more the 80% for both IgL and IgH band by Image Lab software (Bio-Rad).

Avidity Assay Using Live Virus/APS Biosensor

Avidity assays were measured by Octet 96 red using APS biosensor (Forte Bio). APS biosensors were hydrated with D-PBS at least 10 min before an analysis. 5 ug/mL Dengue Live virus serotype 3 in PBS was captured with APS biosensor for 600 sec and 1% BSA PBS were blocked on the surface for 600 sec. 250 ug/mL anti-DENV polyclonal antibodies purified from DEN203 patients' sera in 1% BSA PBS were bound to the biosensor to 1800 sec and then the sensors were incubated in 1% BSA PBS for 1200 sec to dissociate the binding antibody. These reactions were conducted at 30° C. and assay plate, 200 uL/well, were shaken for 1000 rpm. For the negative subtractions, a double subtraction protocol was applied with a combination of antibody/DENV Live virus, antibody/no DENV Live virus, no antibody/DENV Live virus and no antibody/no DENV Live virus to assess the dissociation rate precisely.

Results

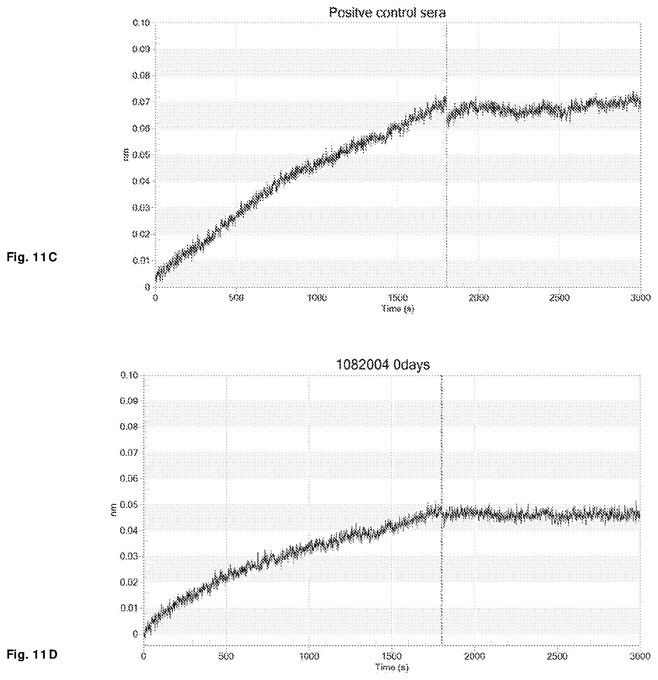

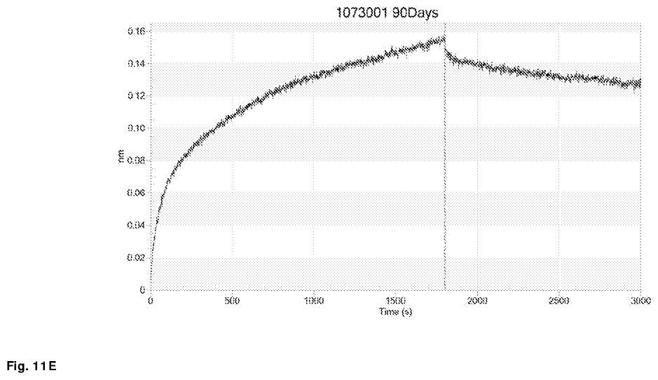

We conducted the avidity assay using Live virus serotype 3 and negative and positive control sera and DEN203 clinical trial samples. All sera were purified IgG by Protein G sepharose to reduce non-specific bindings. There were no signals from IgG negative sera, however, for positive sera and DEN203 samples were observed binding antibody to live virus and antibody dissociation. (FIG. 11)

### Example 6: Avidity Assay of Zika Vaccine Immunized Patients

Materials and Methods

Materials

Zika Virus Like particle (VLP) strain Suriname Z1106033 (GenBank: ALX35659.1) were purchased from Native antigen. SA-Biosensor, biosensor coated with Streptavidin, was purchased from Forte Bio. Negative sera ID 55 51766 P2 and positive sera ID 1043-TDS-0485 were obtained from clinical trials.

Antibody Purification

IgG were purified from these 200 uL sera by Protein G Sepharose (GE). Briefly, 200 uL of sera were mixed with 3 mL D-PBS and 0.6 mL 50% Protein G Sepharose in 15 mL centrifuge tube. These centrifuged tubes were mixed for 90 min at room temperature with shaker. After centrifuging, Protein G Sepharose slurry was transferred to 24 well Unifilter (GE) and the gel washed with D-PBS for 4 time and eluted with 0.1M Glycine HCl pH2.7 for 4 times. The eluates were immediately neutralized to pH 7.0 to 7.5 with 1M Tris HCl pH8.0. The solution was buffer-exchanged using Amicon Ultra 4 (Millipore MWCO 30 KDa). These antibody solutions were measured A280 by NanoDrop 2000 (Thermo) and the IgG concentration calculated. Then these samples were diluted with D-PBS to 2.5 mg/mL for each sample ID. The antibody purity was confirmed by SDS PAGE (NuPAGE 4-12% Bis-Tris Gel, Thermo). 2 ug protein sample was reduced at 70° C. for 10 min and applied to the gel. These gels were stained by simple stain blue (Thermo) and these images were captured by ChemoDoc Touch imaging system (Bio-Rad) and calculated purity was confirmed more the 80% from both IgL and IgH band by Image Lab software (Bio-Rad).

Biotinylation of Zika VLP

50 ug of Zika Virus Like Particle, VLP (Native Antigen) was biotinylated with 50 excess moles of EZ-Link Sulfo-NHS-Biotin (Thermo) following the manufacturer's protocol. The reaction was carried out at room temperature for 60 min, after the reaction, the excess biotinylation reagents was removed and the biotinylated VLPs buffer-exchanged with D-PBS using Amicon Ultra 4 (Millipore MWCO 30 KDa). The biotinylated Zika VLP solutions were adjusted in concentration to 0.5 mg/mL and were stored at 4° C. until use.

Avidity Assay

Avidity assays were measured by Octet HTX (Forte Bio) using SA biosensor (Forte Bio). Briefly, SA biosensors were hydrated with 0.1% BSA PBS-T at least 10 min before an analysis. 5 ug/mL biotinylated Zika VLP, in 0.1% BSA PBS-T were captured SA biosensor and then 50 ug/mL Biocytin (Thermo) was blocked with excess streptavidin on the surface. 250 ug/mL anti-Zika polyclonal antibodies purified from Clinical trial patients' sera in 0.1% BSA PBS-T was bound to the biosensor for 1800 sec and then the sensors were incubated in 0.1% BSA PBS-T 0.35M NaCl for 1200 sec to dissociate the binding antibody. These reactions were conducted at 30° C. and assay plate, 200 uL/well, were shaken for 1000 rpm. For the negative subtractions, double subtraction protocol was applied with a combination of antibody/ZIKA VLP, antibody/no ZIKA VLP, no antibody/ZIKA VLP and no antibody/no ZIKA VLP to assess the dissociation rate precisely. Data analysis was conducted using Octet Data Analysis Software (Forte Bio, version 9.0.0.10) For evaluation, there are two parameters for antibody avidity, Response, correlated with anti-ZIKA antibody concentration, was measured by the response values at 1800 sec association time. k_{off}, antibody dissociation rate, showing strength of antibody binding, was measured by 1:1 binding model fitting from 30 to 600 sec for dissociation. If the dissociation rate of some of the samples could not be measured because of too low dissociation, in this case, the dissociation rate was extrapolated to 2.0×10⁻⁵, detectable dissociation from 0 to 1200 sec for 5% signal decrease. Avidity index was calculated by the following equation; Avidity index=response/k_{off}. The assay was measured twice for each sample showing average values.

Results

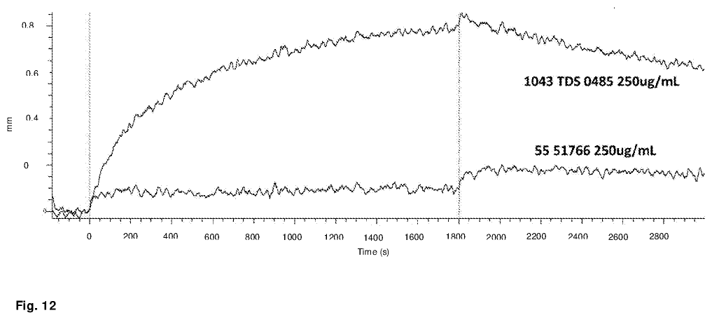

The biosensor gram of Zika Avidity assay is shown in FIG. 12. For IgG purified from negative sera, ID 55 51766 P2 did not bind to biotinylated Zika VLP/SA biosensor. However, for IgG purified from positive sera, ID 1043-TDS-0485, can bind the biosensor and dissociated from the sensors. Avidity index for negative IgG is 5 (response; 0.01 (nm), k_{off} 5.1×10⁻⁴ (−s); Avidity index is 5 (nm*s)). On the other hand, the avidity index for positive sera was 475 (Response; 0.075 (nm), k_{off}; 1.58×10⁻⁴ (−s); Avidity index is 475 (nm*s). these results demonstrate that the avidity assay can be used for determining Zika virus specific antibodies from patient sera.

### Example 7: Avidity Assay of Noro Vaccine Immunized Patients

Materials and Methods

Materials

Noro Virus Like particle (VLP) strain GII.4 Sydney 2012 (GenBank: AMJ17500.1) was prepared in house. SA-Biosensor, biosensor coated with Streptavidin, was purchased from Forte Bio. Negative sera, BRH1540413, and positive sera, BRH1439733, were obtained from clinical trials.

Antibody Purification

IgG was purified from these 200 uL sera by Protein G Sepharose (GE). Briefly, 200 uL of sera were mixed with 3 mL D-PBS and 0.6 mL 50% Protein G Sepharose in 15 mL centrifuge tube. These centrifuged tubes were mixed for 90 min at room temperature with shaker. After centrifuging, Protein G Sepharose slurry was transferred to 24 well Unifilter (GE) and the gel was washed with D-PBS for 4 times and eluted with 0.1M Glycine HCl pH2.7 for 4 times. The eluates were immediately neutralized to pH 7.0 to 7.5 with 1M Tris HCl pH8.0. The solution was buffer-exchanged using Amicon Ultra 4 (Millipore MWCO 30 KDa). These antibody solutions were measured at A280 by NanoDrop 2000 (Thermo) and the IgG concentration was calculated. Then, these samples were diluted with D-PBS to 2.5 mg/mL for each sample ID. The antibody purity was confirmed by SDS PAGE (NuPAGE 4-12% Bis-Tris Gel, Thermo) 2 ug protein samples were reduced at 70° C. for 10 min and applied to the gel. These gels were stained by simple stain blue (Thermo) and these images captured by ChemoDoc Touch imaging system (Bio-Rad) and calculated purity was confirmed more than 80% from IgL and IgH band by Image Lab software (Bio-Rad).

Biotinylation of Noro VLP

50 ug of Noro Virus Like Particle (VLP) is biotinylated with 50 excess moles of EZ-Link Sulfo-NHS-Biotin (Thermo) following the manufacturer's protocol. The reaction is carried out at room temperature for 60 min, after the reaction, the excess biotinylation reagents were removed and these biotinylated VLPs buffer-exchanged with D-PBS using Amicon Ultra 4 (Millipore MWCO 30 KDa). The biotinylated Noro VLP solutions were adjusted in its concentration to 0.5 mg/mL and stored at 4° C. until use.

Avidity Assay

Avidity assay was measured by Octet HTX (Forte Bio) using SA biosensor (Forte Bio). Briefly, SA biosensors were hydrated with 0.1% BSA PBS-T at least 10 min before an analysis. 5 ug/mL biotinylated Noro VLP, in 0.1% BSA PBS-T were captured by SA biosensor and then 50 ug/mL Biocytin (Thermo) was blocked with excess streptavidin on the surface. 125 ug/mL anti-Noro polyclonal antibodies purified from Clinical trial patients' sera in 0.1% BSA PBS-T were bound to the biosensor for 1800 sec and then the sensors were incubated in 0.1% BSA PBS-T 0.35M NaCl for 1200 sec to dissociate the binding antibody. These reactions were conducted at 30° C. and the assay plate was shaken at 1000 rpm. For the negative subtractions, double subtraction protocol was applied with a combination of antibody/Noro VLP, antibody/no Noro VLP, no antibody/Noro VLP and no antibody/no Noro VLP to assess the dissociation rate precisely. Data analysis was conducted by Octet Data Analysis Software (Forte Bio, version 9.0.0.10). For evaluation, there were two parameters for antibody avidity used, Response, correlated with anti-Noro antibody concentration, was measured by the response values at 1800 sec association time. k_{off}, antibody dissociation rate, showing strength of antibody binding, was measured by 1:1 binding model fitting from 30 to 600 sec for dissociation. In case the dissociation rate for some of the samples cannot be measured because of too low dissociation, in this case, the dissociation rate was extrapolated to 2.0×10⁻⁵, detectable dissociation from 0 to 1200 sec for 5% signal decrease. Avidity index was calculated by the following equation; Avidity index=response/k_{off}. The assay was measured twice for each sample showing average values.

Results

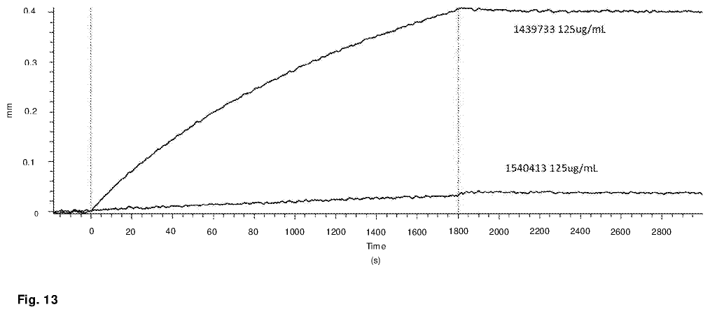

The biosensor gram of Noro Avidity assay is shown in FIG. 13. For IgG purified from negative sera, BRH1540413 weakly bound to biotinylated Noro VLP/SA biosensor. However, for IgG purified from positive sera, BRH1439733 could bind to the biosensor and dissociated from the sensors. Avidity index for negative sera IgG was 1613 (response; 0.032 (nm), k_{off}2×10−5 (−s); Avidity index is 1613 (nm*s)). On the other hand, the avidity index for positive sera was 21681 (Response; 0.403 (nm), k_{off}, 1.99×10−5 (−s); Avidity index was 21681 (nm*s). These results demonstrate that the avidity assay can be used for determining Norovirus specific antibodies from patient sera.

1. A method for determining affinity, binding kinetics and/or concentration of an antibody or of an antibody mixture specific for a virus comprising the following steps:
a) providing a virus-like particle (VLP) attached to a biosensor, wherein said VLP comprises structural proteins from said virus;
b) contacting the VLP attached to the biosensor with a first solution containing the antibody or antibody mixture specific for the virus such that the antibody or antibody mixture binds to the VLP attached to the biosensor and measuring the association of the binding complex;
c) contacting the VLP attached to the biosensor having bound the antibody or antibody mixture with a second solution lacking the antibody or antibody mixture such that the antibody or antibody mixture dissociates from the VLP attached to the biosensor and measuring the dissociation of the binding complex, wherein the measuring in steps b) and c) are performed by surface plasmon resonance (SPR) or biolayer interferometry (BLI); and
d) calculating the affinity, binding kinetics and/or concentration of the antibody or the antibody mixture specific for the virus from the measurement data in steps b) and c).

2. The method according to claim 1, wherein steps b) and c) are performed by BLI.

3. The method of claim 1 or 2, wherein the virus is a flavivirus or a calicivirus and the VLP is derived from said flavivirus or said calicivirus.

4. The method of claim 3, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

5. The method of claim 4, wherein the virus is selected from any of the Dengue virus subtypes DENV-1, DENV-2, DENV-3 and DENV-4.

6. The method of any one of claims 1 to 5, wherein the VLP is attached to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

7. The method of claim 6, wherein the pair of binding molecules is selected from biotin/streptavidin; ligand/receptor; antigen/antibody; antibody/Protein A or Protein G; sugar/lectin; his-tag/Ni and sense/antisense oligonucleotides, preferably the pair of binding molecules is biotin/streptavidin.

8. The method of claim 6 or 7, wherein the VLP is biotinylated and the biosensor has streptavidin attached to its surface.

9. The method claim 6 or 7, wherein the VLP is covalently linked to a biosensor having an amine-reactive surface.

10. The method of any one of claims 1 to 9, wherein steps b) and c) comprise the following steps:
i) contacting the VLP attached to the biosensor with a composition containing the antibody or the antibody mixture under conditions allowing association of the VLP with the antibody or antibody mixture; and
ii) removing the VLP attached to the biosensor from the composition containing the antibody or the antibody mixture and incubating the VLP attached to the biosensor under conditions allowing the dissociation of the VLP from the antibody or antibody mixture.

11. A method for determining affinity, binding kinetics and/or concentration of an antibody or of an antibody mixture specific for a virus comprising the following steps:
a) providing a live virus or an inactivated virus attached to a biosensor;
b) contacting the live virus or inactivated virus attached to the biosensor with a first solution containing the antibody or antibody mixture specific for the virus such that the antibody or antibody mixture binds to the live virus or inactivated virus attached to the biosensor and measuring the association of the binding complex;
c) contacting the live virus or inactivated virus attached to the biosensor having bound the antibody or antibody mixture with a second solution lacking the antibody or antibody mixture such that the antibody or antibody mixture dissociates from the live virus or inactivated virus attached to the biosensor and measuring the dissociation of the binding complex, wherein the measuring in steps b) and c) are performed by SPR or BLI; and
d) calculating the affinity, binding kinetics and/or concentration of the antibody or the antibody mixture specific for the virus.

12. The method according to claim 11, wherein steps b) and c) are performed by BLI.

13. The method of claim 10, wherein said live virus or said inactivated virus is a flavivirus or a calicivirus.

14. The method of claim 13, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

15. The method of claim 14, wherein the virus is selected from any of the Dengue virus subtypes DENV-1, DENV-2, DENV-3 and DENV-4.

16. The method of claim any one of claims 11 to 15, wherein said live virus or said inactivated virus is attached to the biosensor by hydrophobic interaction of said live virus or said inactivated virus with a capture reagent linked to the surface of the biosensor.

17. The method of claim 16, wherein the capture reagent comprises aminopropylsilane.

18. The method of any one of claims 11 to 17, wherein steps b) and c) comprise the following steps:
i) contacting said live virus or said inactivated virus with a composition containing said antibody or said antibody mixture under conditions allowing association of said live virus or said inactivated virus with the antibody or antibody mixture; and
ii) removing said live virus or said inactivated virus from the composition containing said antibody or said antibody mixture and incubating said live virus or said inactivated virus under conditions allowing the dissociation of said live virus or said inactivated virus from said antibody or said antibody mixture.

19. The method of any one of claims 1 to 18, wherein said antibody mixture is a polyclonal mixture of antibodies from serum of a human subject immunized with the virus or a virus antigen thereof.

20. The method of claim 19, wherein said antibody or said antibody mixture is purified by affinity chromatography, preferably the affinity chromatography comprises the use of Protein A, Protein G, Protein A/G or Protein L affinity chromatography.

21. The method of any one of claims 1 to 20, wherein the method is used for determining the avidity of the antibody or antibody mixture for the virus, preferably the avidity index is determined.

22. A method for determining the avidity and/or affinity over time of an antibody or antibody mixture produced after immunization of a human subject with a virus vaccine comprising the following steps:
c) obtaining serum samples from said subject at different time points after immunization;
d) purifying the antibody or antibody mixture from the serum samples by affinity chromatography using Protein A Protein G, Protein A/G or Protein L;
e) determining the avidity and/or affinity of the antibodies for the virus as a function over time in accordance with the method of any one of claims 1 to 21.

23. The method according to claim 22, wherein the method determines the avidity index of the antibody or antibody mixture from serum samples obtained after different points of time after immunization.

24. The method according to claim 23, wherein the virus is selected from Dengue virus, Japanese encephalitis virus, Tick-borne encephalitis virus, West Nile virus, Yellow fever, Zika virus and Norovirus, preferably, the virus is Dengue virus, Zika virus or Norovirus.

25. The method according to any one of claims 22 to 24, wherein the virus vaccine is a tetravalent dengue virus composition comprising four live, attenuated dengue virus strains.

26. The method according to claim 25, wherein the four live, attenuated dengue virus strains are:
(i) a chimeric dengue serotype 2/1 strain, preferably the chimeric serotye 2/1 strain has the amino acid sequence set forth in SEQ ID NO: 2,
(ii) a dengue serotype 2 strain, preferably the serotype 2 strain has the amino acid sequence set forth in SEQ ID NO: 4,
(iii) a chimeric dengue serotype 2/3 strain, preferably the chimeric serotype 2/3 strain has the amino acid sequence set forth in SEQ ID NO: 6, and
(iv) a chimeric dengue serotype 2/4 strain, preferably the chimeric serotype 2/4 strain has the amino acid sequence set forth in SEQ ID NO: 8.

27. The method according to claim 25 or 26, wherein each one of the four live, attenuated dengue virus strains has attenuating mutations in the 5′-noncoding region (NCR) at nucleotide 57 from cytosine to thymine, in the NS1 gene at nucleotide 2579 from guanine to adenine resulting in an amino acid change at position 828 of the NS1 protein from glycine to asparagine, and in the NS3 gene at nucleotide 5270 from adenine to thymine resulting in an amino acid change at position 1725 of the NS3 protein from glutamine to valine.

28. A method of preparing a virus-like particle (VLP) attached to a biosensor suitable for SPR or BLI, wherein said VLP comprises structural proteins from said virus, wherein the method comprises attaching the VLP to the biosensor by any of the following:
i) a pair of binding molecules capable of specifically binding to each other, wherein the first binding molecule is linked to the VLP and the second binding molecule is attached to the surface of the biosensor; and/or
ii) a covalent linkage of the VLP to a capture reagent attached to the biosensor.

29. The method according to claim 28, wherein the virus is a flavivirus or a calicivirus, preferably the virus is selected from Dengue virus, Japanese encephalitis virus, Tick-borne encephalitis virus, West Nile virus, Yellow fever virus, Zika virus and Norovirus.

30. A VLP attached to a biosensor suitable for SPR or BLI obtainable by the method according to claim 28 to 29.

31. A method of preparing a live virus or an inactivated virus attached to a biosensor suitable for SPR or BLI, wherein the method comprises attaching said live virus or said inactivated virus to the biosensor by hydrophobic interaction of said live virus or said inactivated virus with a capture reagent linked to the surface of the biosensor.

32. The method according to claim 31, wherein the virus is a flavivirus or a calicivirus, preferably the virus is selected from Dengue virus, Japanese encephalitis virus, Tick-borne encephalitis virus, West Nile virus, Yellow fever virus, Zika virus and Norovirus.

33. A live virus or inactivated virus attached to a biosensor suitable for SPR or BLI obtainable by the method according to claim 31 or 32.
